(19) United States
(12) Patent Application Publication
(10) Pub. No.: US 2011/0201642 A1
(43) Pub. Date: Aug. 18, 2011

(54) Bioactive Compounds

(76) Inventors: John Blunt, Christchurch (NZ); Tony Cole, Christchurch (NZ); Murray Munro, Christchurch (NZ); Lin Sun, Christchurch (NZ); Jean-Frederic rene Weber, Malaysia (MY); Kalavathy Ramasamy, Malaysia (MY); Hamidah Abu Bakar, Malaysia (MY); Abu Bakar Bin Abdul Majeed, Malaysia (MY)

(21) Appl. No.: 13/126,292

(22) PCT Filed: Oct. 30, 2009
(86) PCT No.: PCT/MY2009/000176

§ 371 Date: Apr. 27, 2011

(30) Foreign Application Priority Data
Nov. 3, 2008 (NZ) 572489

(51) Int. Cl. A61K 31/4353; C07D 471/04; A61P 35/00

(52) U.S. Cl. 514/300; 540/460

(57) ABSTRACT

The invention relates to bioactive compounds derived from an endophytic Aspergillus sp. fungus strain isolated from a Malaysian medicinal plant Garcinia scortechinii and to compositions which contain one or more of these compounds. In particular, the invention relates to compounds according to formula I; or a pharmaceutically acceptable salt, solvate, hydrate or prodrug derivative thereof, as pure stereoisomers, mixture of isomers, in enol form or tautomeric form. These compounds have utility in, for example, anti-cancer treatments.

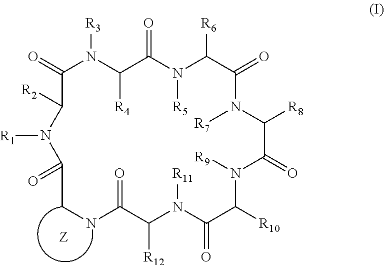

### TECHNICAL FIELD

This invention relates to bioactive compounds and to compositions which contain one or more of these compounds. In particular, the invention relates to compounds which have cytotoxic properties. These compounds have utility in, for example, anti-cancer treatments.

### BACKGROUND ART

Compounds from both terrestrial and marine natural sources have displayed useful anti-cancer activity and have proved to be successful in clinical trials.

Cyclic peptides and depsipeptides are constantly being discovered from natural sources. Examples of these compounds include cyclosporin A (immunosuppressive), a very effective drug, and kahalalide F, a promising anti-cancer drug candidate currently undergoing phase 2/3 trials.

The applicants have now identified a series of bioactive compounds from an endophytic Aspergillus sp. fungus strain isolated from Garcinia scortechinii, a Malaysian medicinal plant. The compounds have cytotoxic properties. This invention is broadly directed towards this fungus, the compounds and structurally related analogues, and to compositions, uses and methods of treatment that employ these compounds.

Accordingly, it is an object of the present invention to provide compounds having cytotoxic properties, and compositions comprising same, and/or to at least provide the public with a useful choice.

Other objects of the invention may become apparent from the following description which is given by way of example only.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date.

### SUMMARY OF THE INVENTION

In a first aspect, the present invention provides Aspergillus sp. NMI No. V08/027,588.

In a further aspect, the present invention provides a biologically pure culture of an Aspergillus sp. strain on deposit at National Measurement Institute, Pymble, Australia, under accession No. V08/027,588 or a culture having the identifying characteristics thereof.

In another aspect, the present invention provides a compound of Formula I, or a pharmaceutically acceptable salt, solvate, hydrate or prodrug derivative thereof:

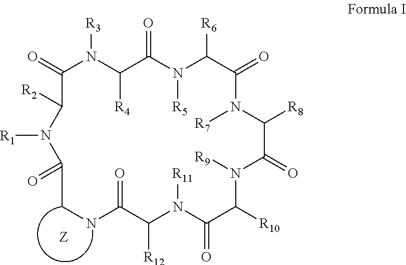

comprising of:
R₁, R₃, R₅, R₇, R₉ and R₁₁, which are each independently selected from the group consisting of —H, alkyl, substituted alkyl and —(C═O)R; wherein R is selected from the group consisting of alkyl, substituted alkyl, cycloalkyl, substituted cycloalkyl, alkenyl, substituted alkenyl, cycloalkenyl, substituted cycloalkenyl, alkynyl, substituted allynyl, aryl, substituted aryl, heteroaryl, substituted heteroaryl, heterocyclyl and substituted heterocyclyl; and
R₂, R₄, R₆, R₈, R₁₀ and R₁₂, which are each independently selected from the group consisting of alkyl, substituted alkyl, alkenyl and substituted alkenyl;
wherein each substituted alkyl, substituted cycloalkyl, substituted alkenyl, substituted cycloalkenyl, substituted alkynyl, substituted aryl, substituted heteroaryl, and/or substituted heterocyclyl has 1-3 substituents each independently selected from the group consisting of: —OH, —OR′, —SH, —SR′, —SOR′, —SO₂R′, —NO₂, —NH₂, —NHR′, —N(R′)₂, —NHCOR′, —N(COR′)₂, —NHSO₂R′, —CN, halogen, —C(═O)H, —C(═O)R′, —CO₂H, —CO2R′, alkyl, alkyl substituted with 1-3 R″, alkenyl, alkenyl substituted with 1-3 R″, cycloalkenyl, cycloalkenyl substituted with 1-3 R″, alkynyl, alkynyl substituted with 1-3 R″, aryl, aryl substituted with 1-3 R″, heterocyclyl, heterocyclyl substituted with 1-3 R″, heteroaryl and heteroaryl substituted with 1-3 R″;
the Z ring, which is selected from the group consisting of:

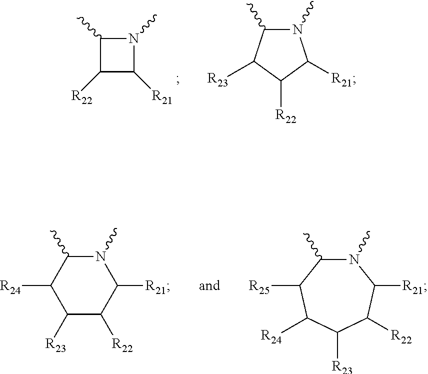

wherein R₂₁, R₂₂, R₂₃, R₂₄ and R₂₅ are each independently selected from the group consisting of: —H, —OH, —OR′, —SH, —SR′, —SOR′, —SO₂R′, —NO₂, —NH₂, —NHR′, —N(R′)₂, —NHCOR′, —N(COR′)₂, —NHSO₂R′, —CN, halogen, —C(═O)H, —C(═O)R′, —CO₂H, —CO₂R′, alkyl, alkyl substituted with 1-3 R″, alkenyl, alkenyl substituted with 1-3 R″, cycloalkenyl, cycloalkenyl substituted with 1-3 R″, alkynyl, alkynyl substituted with 1-3 R″, aryl, aryl substituted with 1-3 R″, heterocyclyl, heterocyclyl substituted with 1-3 R″, heteroaryl and heteroaryl substituted with 1-3 R″; and
wherein each R′ is independently selected from the group consisting of alkyl, alkyl substituted with 1-3 R″, cycloalkyl, cycloalkyl substituted with 1-3 R″, alkenyl, alkenyl substituted with 1-3 R″, cycloalkenyl, cycloalkenyl substituted with 1-3 R″, alkynyl, alkynyl substituted with 1-3 R″, aryl, aryl substituted with 1-3 R″, alkylaryl, alkylaryl substituted with 1-3 R″, heterocyclyl, heterocyclyl substituted with 1-3 R″, heteroaryl and heteroaryl substituted with 1-3 R″; wherein each R″ is independently selected from the group consisting of: —OH, —SH, —NO₂, —NH₂, —CN, halogen, —C(═O)H, and —CO₂H.

In another aspect, the present invention provides a method for the production of a compound of Formula I which involves isolating the compound from a natural source.

In another aspect, the present invention provides a compound of Formula I obtainable from a culture of an Aspergillus sp. strain on deposit at National Measurement Institute, Pymble, Australia, under accession No. V08/027,588 or a culture having the identifying characteristics thereof.

In another aspect, the present invention provides a compound of Formula I for use as a medicament.

In another aspect, the present invention provides a method for the treatment or prophylaxis of cancer or another disease in a mammal comprising the step of administering a therapeutically effective amount of a compound of Formula I to the mammal.

In another aspect, the present invention provides a use of a compound of Formula I for the manufacture of a medicament for treating cancer or another disease.

In another aspect, the invention provides a composition comprising a compound of Formula I. In a preferred embodiment, the composition is a pharmaceutical composition and further comprises a pharmaceutically acceptable carrier, diluent or excipient.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Although the present invention is broadly as defined above, those persons skilled in the art will appreciate that the invention is not limited thereto and that the invention also includes embodiments of which the following description gives examples.

### BRIEF DESCRIPTION OF THE DRAWINGS

The invention will now be described with reference to the Figures in which:

FIGS. 1(a) to (e) shows the structural formulae of Compounds A1-A5 obtained from the Aspergillus sp. strain NMI No. V08/027,588.

FIG. 2 shows the stereochemical structure of Compound A2 obtained from the Aspergillus sp. strain NMI No. V08/027,588.

FIG. 3 shows the stereochemical structure of Compound A3 obtained from the Aspergillus sp. strain NMI No. V08/027,588.

FIG. 4 shows the partial stereochemistry of a compound of Formula Ia.

### DETAILED DESCRIPTION OF THE INVENTION

As described above, this invention is directed to new bioactive compounds. Several of these compounds has been isolated from a new fungal strain—Aspergillus sp. —that was obtained from Garcinia scortechinii, a Malaysian medicinal plant. These compounds have inter alia cytotoxic properties.

In one aspect, the present invention is directed to a strain of Aspergillus sp. from which the new bioactive compounds were isolated.

The new Aspergillus sp. strain has been deposited in the National Measurement Institute Laboratories (NMI), Suakin Street, Pymble, New South Wales, Australia on 27 Oct. 2008 according to the Budapest Treaty for the purposes of patent procedure. The deposited strain has been accorded the deposit number V08/027,588.

Details of the isolation and selection process employed to obtain the deposited Aspergillus sp. strain are set out in the Examples. Identifying morphological characteristics of the deposited Aspergillus sp. strain are also provided in the Examples.

The applicants are the first to provide Aspergillus sp. strain NMI No. V08/027,588 in isolated form.

Accordingly, in one aspect the invention provides Aspergillus sp. NMI No. V08/027,588.

Also contemplated herein are Aspergillus sp. strains having the identifying characteristics of Aspergillus sp. NMI No. V08/027,588 as set forth in the examples. These strains may be mutants which are natural products or artificially produced by manipulations such as chemical or UV mutagenesis, or genetic modification.

In one embodiment, the Aspergillus sp. strain of the invention is isolated. Preferably, the strain is provided in the form of a biologically pure culture.

Accordingly, in another aspect, the invention provides a biologically pure culture of an Aspergillus sp. strain on deposit at National Measurement Institute, Pymble, Australia, under accession No. V08/027,588 or a culture having the identifying characteristics thereof.

The invention also provides compounds that may be isolated from the Aspergillus sp. strain of the invention and derivatives of those compounds.

Accordingly, in another aspect, the invention provides a compound of Formula I, or a pharmaceutically acceptable salt, solvate, hydrate or prodrug derivative thereof:

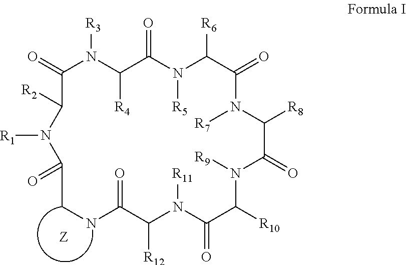

comprising of:
R₁, R₃, R₅, R₇, R₉ and R₁₁, which are each independently selected from the group consisting of: —H, alkyl, substituted alkyl and —(C═O)R; wherein R is selected from the group consisting of alkyl, substituted alkyl, cycloalkyl, substituted cycloalkyl, alkenyl, substituted alkenyl, cycloalkenyl, substituted cycloalkenyl, alkynyl, substituted alkynyl, aryl, substituted aryl, heteroaryl, substituted heteroaryl, heterocyclyl and substituted heterocyclyl; and
R₂, R₄, R₆, R₈, R₁₀ and R₁₂, which are each independently selected from the group consisting of alkyl, substituted alkyl, alkenyl and substituted alkenyl;
wherein each substituted alkyl, substituted cycloalkyl, substituted alkenyl, substituted cycloalkenyl, substituted alkynyl, substituted aryl, substituted heteroaryl, and/or substituted heterocyclyl has 1-3 substituents each independently selected from the group consisting of: —OH, —OR′, —SH, —SR′, —SOR′, —SO₂R′, —NO₂, —NH₂, —NHR′, —N(R)₂, —NHCOR′, —N(COR′)₂, —NHSO₂R′, —CN, halogen, —C(═O)H, —C(═O)R′, —CO₂H, —CO₂R′, alkyl, alkyl substituted with 1-3 R″, alkenyl, alkenyl substituted with 1-3 R″, cycloalkenyl, cycloalkenyl substituted with 1-3 R″, alkynyl, alkynyl substituted with 1-3 R″, aryl, aryl substituted with 1-3 R″, heterocyclyl, heterocyclyl substituted with 1-3 R″, heteroaryl and heteroaryl substituted with 1-3 R″;
the Z ring, which is selected from the group consisting of:

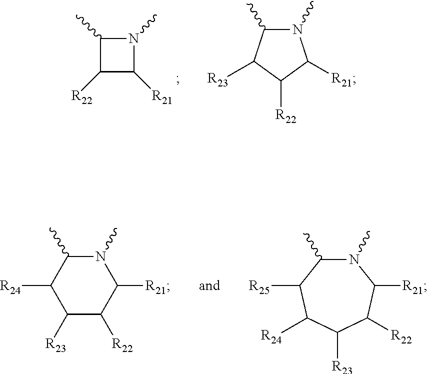

wherein R₂₁, R₂₂, R₂₃, R₂₄ and R₂₅ are each independently selected from the group consisting of: —H, —OH, —OR′, —SH, —SR′, —SOR′, —SO₂R′, —NO₂, —NH₂, —NHR′, —N(R)₂, —NHCOR′, —N(COR′)₂, —NHSO₂R′, —CN, halogen, —C(═O)H, —C(═O)R′, —CO₂H, —CO₂R′, alkyl, alkyl substituted with 1-3 R″, alkenyl, alkenyl substituted with 1-3 R″, cycloalkenyl, cycloalkenyl substituted with 1-3 R″, alkynyl, alkynyl substituted with 1-3 R″, aryl, aryl substituted with 1-3 R″, heterocyclyl, heterocyclyl substituted with 1-3 R″, heteroaryl and heteroaryl substituted with 1-3 R″; and
wherein each R′ is independently selected from the group consisting of alkyl, alkyl substituted with 1-3 R″, cycloalkyl, cycloalkyl substituted with 1-3 R″, alkenyl, alkenyl substituted with 1-3 R″, cycloalkenyl, cycloalkenyl substituted with 1-3 R″, alkynyl, alkynyl substituted with 1-3 R″, aryl, aryl substituted with 1-3 R″, alkylaryl, alkylaryl substituted with 1-3 R″, heterocyclyl, heterocyclyl substituted with 1-3 R″, heteroaryl and heteroaryl substituted with 1-3 R″; wherein each R″ is independently selected from the group consisting of: —OH, —SH, —NO₂, —NH₂, —CN, halogen, —C(═O)H, and —CO₂H.

As used herein, the term “pharmaceutically acceptable salt” is intended to include acid addition salts of any basic moiety that may be present in a compound of Formula I, and base addition salts of any acidic moiety that may be present in a compound of Formula I.

Such salts are generally prepared by reacting the compound with a suitable organic or inorganic acid or base. Examples of pharmaceutically acceptable salts of basic moieties include: sulfates; methanesulfonates; acetates; propionates; caproates; hydrochlorides; hydrobromides; phosphates; toluenesulfonates; citrates; maleates; succinates; tartrates; lactates; valerates; enanthates; cypionates and fumarates. Examples of pharmaceutically acceptable salts of acidic moieties include: ammonium salts; alkali metal salts such as sodium salts and potassium salts; and alkaline earth metal salts such as calcium salts and magnesium salts. Other pharmaceutically acceptable salts will be apparent to those skilled in the art.

As used herein, the term “prodrug derivative” is intended to include functional derivatives of the compounds of Formula I, the pharmacological action of which results from conversion to a compound of Formula I by metabolic processes within the body. Therefore, a prodrug derivative is any covalently bonded carrier that releases a compound of Formula I in vivo when the prodrug derivative is administered to a mammal. Prodrug derivatives are generally prepared by modifying functional groups in such a way that the modification is cleaved in vivo to yield the parent compound. The term prodrug derivative also includes polymeric prodrugs.

The invention also contemplates prodrug derivatives that are converted to a compound of Formula I by a separately administered targeting agent—antibody directed enzyme prodrug therapy (ADEPT). In these embodiments, the inactive prodrug is converted to the compound of Formula I by an enzyme, which is the targeting agent. The enzyme is coupled to an antibody that directs it to the tissue of interest. The prodrug is activated only at the site targeted by the enzyme, which may spare other tissues from potentially toxic side effects.

Conventional procedures for the selection and preparation of suitable prodrug derivatives are known to those persons skilled in the art and are discussed in, for example, T. Higuchi and V. Stella, Pro-drugs as Novel Delivery Systems, volume 14 of the A.C.S. Symposium Series, 1987; E. B. Roche (ed.), Bioreversible Carriers in Drug Design, Pergamon Press, New York, 1987; V. J. Stella et al. (eds), Prodrugs: Challenges and Rewards, Springer, New York, 2007; and R. G. Melton and R. J. Knox (eds), Enzyme-Prodrug Strategies for Cancer Therapy, Springer, New York, 1999.

The compounds of Formula I may form hydrates, or solvates with pharmaceutically acceptable solvents. The present invention contemplates such hydrates and solvates as well as the corresponding unsolvated forms.

The general chemical terms used in Formula I herein have their usual meanings. For example, as used herein:

the term “alkyl” is intended to include straight chain and branched chain saturated hydrocarbon groups. In one embodiment, preferred alkyl groups comprise 1 to 6 carbon atoms. In another preferred embodiment, the alkyl group is methyl, ethyl, n-propyl, iso-propyl, n-butyl, iso-butyl, sec-butyl, tert-butyl, n-pentyl, 1-methylbutyl, 2-methylbutyl, 3-methylbutyl or 2,2′-dimethylpropyl;
the term “alkenyl” is intended to include straight chain or branched chain mono-unsaturated hydrocarbon groups;
the term “aryl” is intended to include aromatic radicals including, but not limited to: phenyl; naphthyl; indanyl; biphenyl; and the like. In one embodiment, preferred aryl groups comprise 4 to 10 carbon atoms;
the term “cycloalkyl” is intended to include cyclic saturated hydrocarbon groups. In one embodiment, preferred cycloalkyl groups comprise 3 to 6 carbon atoms;
the term “cycloalkenyl” is intended to include cyclic mono-unsaturated hydrocarbon groups;
the term “heteroaryl” is intended to include heteroaromatic radicals including, but not limited to: pyrimidinyl; pyridyl; pyrrolyl; furyl; oxazolyl; thiophenyl; and the like; and
the term “heterocyclyl” is intended to include non-aromatic saturated heterocyclic radicals including, but not limited to: piperidinyl; pyrrolidinyl; piperazinyl; 1,4-dioxanyl; tetrahydrofuranyl; tetrahydrothiophenyl; and the like.

As used herein, the term “substituted” is intended to mean that one or more hydrogen atoms in the group indicated is replaced with one or more independently selected substituents, provided that the normal valency of each atom to which the substituents are attached is not exceeded, and that the substitution results in a stable compound.

In a preferred embodiment, R₁, R₃ and R₉ are —H.

In a further preferred embodiment, R₅, R₇, and R₁₁ are alkyl, preferably methyl.

In another preferred embodiment, R₂, R₄, R₆, R₈ and R₁₂ are alkyl or substituted alkyl.

In another preferred embodiment, R₄, R₆, and R₁₂ are alkyl and R₂ and R₈ are substituted alkyl.

In another preferred embodiment, R₆ and R₁₂ are alkyl, preferably methyl.

In another preferred embodiment, R₂ is substituted alkyl, preferably 1-hydroxy-2-methylpropyl.

In another preferred embodiment R₄ is alkyl, preferably iso-propyl or sec-butyl.

In another preferred embodiment, R₈ is substituted alkyl, preferably 1-hydroxy-2-methylpropyl or 1-hydroxy-2-methylbutyl.

In another preferred embodiment, R₁₀ is alkyl or alkenyl.

In another preferred embodiment, R₁₀ is alkyl, preferably iso-butyl or 2-methylbutyl.

In another preferred embodiment, R₁₀ is alkenyl, preferably 2-methyl-3-butenyl.

In another preferred embodiment, the Z ring is:

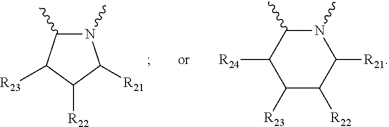

In another preferred embodiment, the Z ring is:

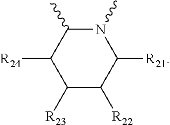

In another preferred embodiment, the Z ring is:

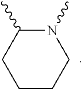

In another preferred embodiment, R₁, R₃ and R₉ are —H; R₅, R₇, and R₁₁ are methyl; R₂ is 1-hydroxy-2-methylpropyl; R₄ is iso-propyl or sec-butyl; R₆ and R₁₂ are methyl; R₈ is 1-hydroxy-2-methylpropyl or 1-hydroxy-2-methylbutyl; R₁₀ is iso-butyl, 2-methylbutyl or 2-methyl-3-butenyl; and the Z ring is:

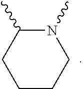

In a particularly preferred embodiment, R₁, R₃ and R₉ are —H; R₅, R₇, and R₁₁ are methyl; R₂ is 1-hydroxy-2-methylpropyl; R₄ is iso-propyl; R₆ and R₁₂ are methyl; R₈ is 1-hydroxy-2-methylpropyl; R₁₀ is 2-methyl-3-butenyl; and the Z ring is:

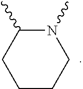

In another particularly preferred embodiment, R₁, R₃ and R₉ are —H; R₅, R₇, and R₁₁ are methyl; R₂ is 1-hydroxy-2-methylpropyl; R₄ is sec-butyl; R₆ and R₁₂ are methyl; R₈ is 1-hydroxy-2-methylpropyl; R₁₀ is iso-butyl; and the Z ring is:

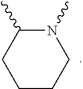

In another particularly preferred embodiment, R₁, R₃ and R₉ are —H; R₅, R₇, and R₁₁ are methyl; R₂ is 1-hydroxy-2-methylpropyl; R₄ is sec-butyl; R₆ and R₁₂ are methyl; R₈ is 1-hydroxy-2-methylpropyl; R₁₀ is 2-methyl-3-butenyl; and the Z ring is:

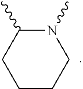

In another particularly preferred embodiment, R₁, R₃ and R₉ are —H; R₅, R₇, and R₁₁ are methyl; R₂ is 1-hydroxy-2-methylpropyl; R₄ is sec-butyl; R₆ and R₁₂ are methyl; R₈ is 1-hydroxy-2-methylpropyl; R₁₀ is 2-methylbutyl; and the Z ring is:

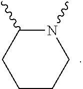

In another particularly preferred embodiment, R₁, R₃ and R₉ are —H; R₅, R₇, and R₁₁ are methyl; R₂ is 1-hydroxy-2-methylpropyl; R₄ is sec-butyl; R₆ and R₁₂ are methyl; R₈ is 1-hydroxy-2-methylbutyl; R₁₀ is 2-methyl-3-butenyl; and the Z ring is:

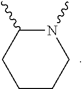

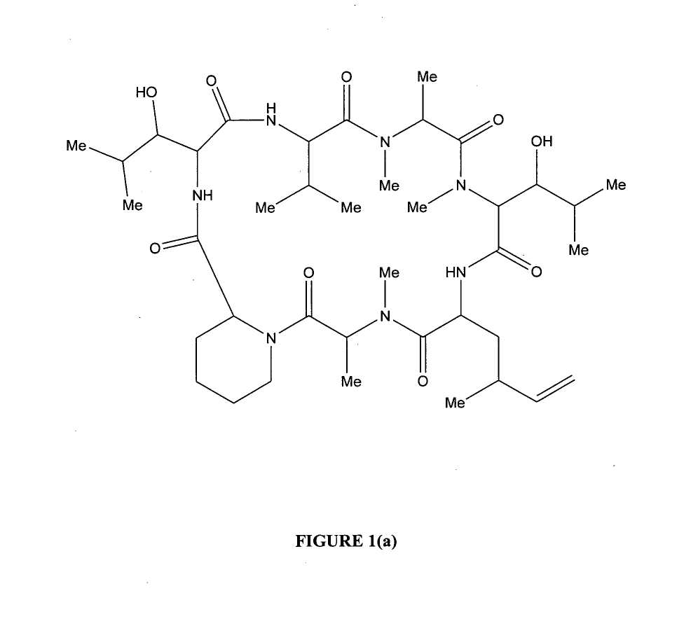

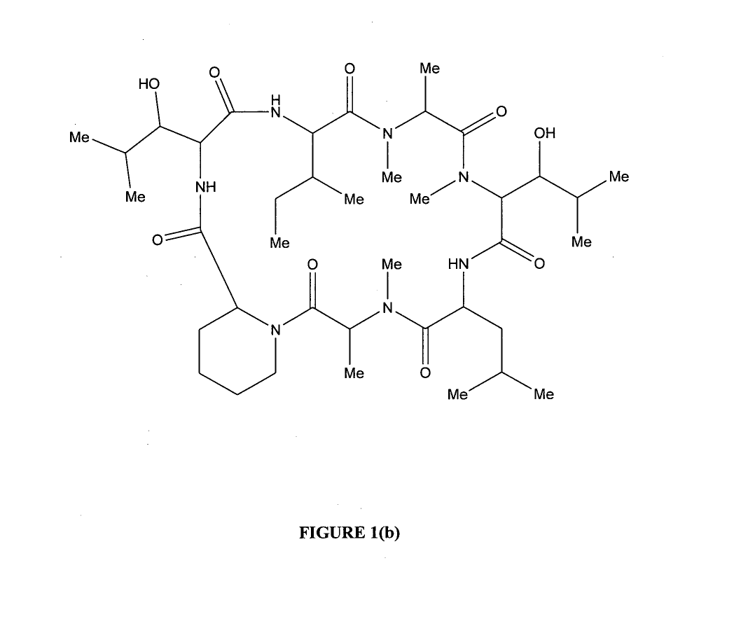

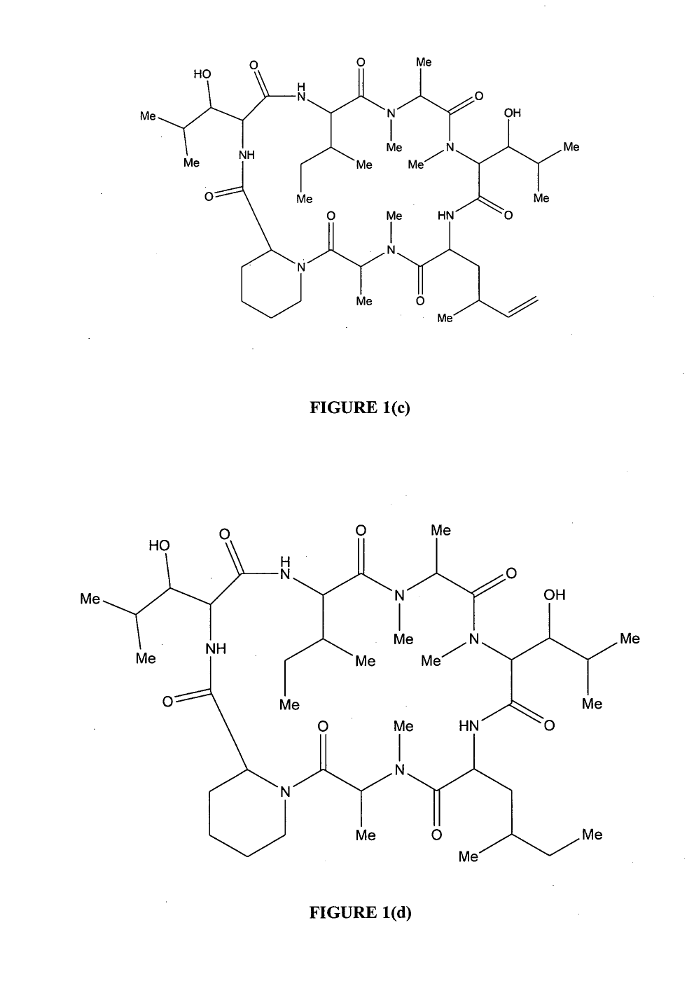

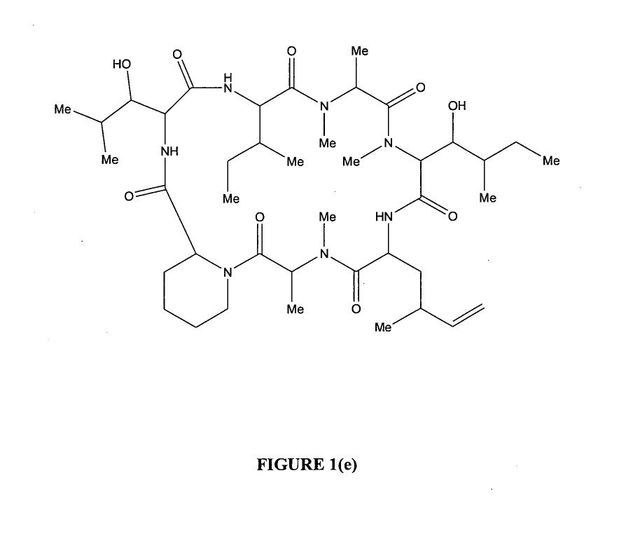

In another particularly preferred embodiment, the compound is one of the compounds having the structures shown in FIGS. 1(a) to 1(e) and FIGS. 2 to 4.

In another preferred embodiment, the compound has the Formula Ia:

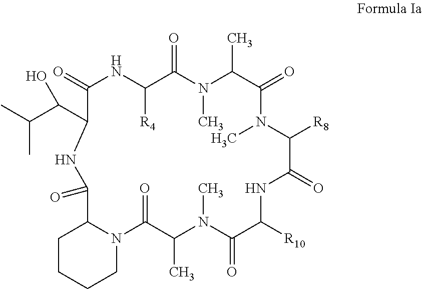

wherein R₄, R₈ and R₁₀ are as defined for Formula I.

In a preferred embodiment of a compound of Formula Ia, R₄ is alkyl and R₈ is substituted alkyl.

In another preferred embodiment of a compound of Formula Ia, R₄ is alkyl, preferably iso-propyl or sec-butyl.

In another preferred embodiment of a compound of Formula Ia, R₈ is substituted alkyl, preferably 1-hydroxy-2-methylpropyl or 1-hydroxy-2-methylbutyl.

In another preferred embodiment of a compound of Formula Ia, R₁₀ is alkyl or alkenyl.

In another preferred embodiment of a compound of Formula Ia, R₁₀ is alkyl, preferably iso-butyl or 2-methylbutyl.

In another preferred embodiment of a compound of Formula Ia, R₁₀ is alkenyl, preferably 2-methyl-3-butenyl.

In a particularly preferred embodiment of a compound of Formula Ia, R₄ is iso-propyl, R₈ is 1-hydroxy-2-methylpropyl and R₁₀ is 2-methyl-3-butenyl.

In another particularly preferred embodiment of a compound of Formula Ia, R₄ is sec-butyl, R₈ is 1-hydroxy-2-methylpropyl and R₁₀ is iso-butyl.

In another particularly preferred embodiment of a compound of Formula Ia, R₄ is sec-butyl, R₈ is 1-hydroxy-2-methylpropyl and R₁₀ is 2-methyl-3-butenyl.

In another particularly preferred embodiment of a compound of Formula Ia, R₄ is sec-butyl, R₈ is 1-hydroxy-2-methylpropyl and R₁₀ is 2-methylbutyl.

In another particularly preferred embodiment of a compound of Formula Ia, R₄ is sec-butyl, R₈ is 1-hydroxy-2-methylbutyl and R₁₀ is 2-methyl-3-butenyl.

In a further aspect, the invention provides a compound having the ¹H NMR and/or ¹³C NMR spectral data shown in any one of Tables 6 to 10 in the Examples.

The compounds of the invention have asymmetric carbon atoms. Therefore, stereoisomers (both enantiomers and diastereomers) of such compounds can exist. The present invention contemplates the pure stereoisomers and any mixture of the isomers. For example, a pure enantiomer of a compound of the invention can be isolated from a mixture of enantiomers of the compound using conventional optical resolution techniques. Enol forms and tautomers, where appropriate, are also contemplated.

In a preferred embodiment of a compound of Formula Ia, the compound has a partial stereochemical structure of:

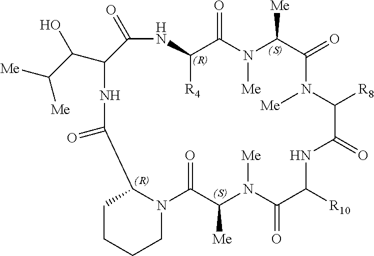

wherein R₄, R₈ and R₁₀ are as defined for Formula I.

The invention also provides a method for the production of a compound of Formula I that involves isolating the compound from a natural source or synthesising the compound by chemical means.

The compounds of Formula Ia can be prepared by isolating the compound from a natural source. In particular, these compounds can be obtained from the Aspergillus sp. strain of the invention. The compounds can be isolated by extracting the fungus with a suitable solvent.

In one embodiment, the solvent is ethyl acetate.

A preferred extraction process is described in the Examples.

Accordingly, in another aspect, the present invention provides a compound of Formula I obtainable from a culture of an Aspergillus sp. strain on deposit at National Measurement Institute, Pymble, Australia, under accession No. V08/027,588 or a culture having the identifying characteristics thereof.

Other compounds of the present invention may be prepared by, for example, reacting the compounds of Formula Ia; wherein R₄ is iso-propyl, R₈ is 1-hydroxy-2-methylpropyl and R₁₀ is 2-methyl-3-butenyl; R₄ is sec-butyl, R₈ is 1-hydroxy-2-methylpropyl and R₁₀ is iso-butyl; R₄ is sec-butyl, R₈ is 1-hydroxy-2-methylpropyl and R₁₀ is 2-methyl-3-butenyl; R₄ is sec-butyl, R₈ is 1-hydroxy-2-methylpropyl and R₁₀ is 2-methylbutyl; or R₄ is sec-butyl, R₈ is 1-hydroxy-2-methylbutyl and R₁₀ is 2-methyl-3-butenyl; with suitable reagents to produce derivatives. Sequential reactions may be used to prepare a wide range of derivatives. The selection of suitable reagents and reaction conditions is within the ability of those persons skilled in the art. Protection and deprotection reactions may also be employed in the overall synthetic strategy in order to obtain the desired derivative.

Reactions that are particularly contemplated for preparing derivatives from the compounds of Formula Ia include, but are not limited to: hydroxylation; dihydroxylation; oxidation; reduction; hydrogenation; epoxidation; acylation; and substitution.

In other embodiments, the compounds of the invention may be prepared from suitable D- or L-configuration alpha-amino acids by conventional peptide synthesis techniques.

In a preferred embodiment, a compound of the invention may be prepared by a method comprising the steps of:
(a) attaching a suitably protected alpha-amino acid to a resin;
(b) deprotecting the alpha-amino acid;
(c) coupling another suitably protected alpha-amino acid to the deprotected amino acid;
(d) repeating steps (b) and (c) until the desired acyclic polypeptide is obtained;
(e) optionally protecting the acyclic polypeptide;
(f) cleaving the acyclic polypeptide from the resin; and
(g) cyclising the acyclic polypeptide to obtain the compound of the invention.

In one embodiment, the suitably protected alpha-amino acids are selected from the group consisting of protected analogues of: pipecolic acid; 3-hydroxyleucine; valine; isoleucine; N-methylalanine; N-methyl-3-hydroxyleucine; 2-amino-4-methyl-5-hexenoic acid; leucine; and 2-amino-4-methylhexanoic acid.

In a preferred embodiment, the suitably protected alpha-amino acids are selected from the group consisting of protected analogues of: pipecolic acid; 3-hydroxyleucine; valine; N-methylalanine; N-methyl-3-hydroxyleucine; and 2-amino-4-methyl-5-hexenoic acid.

In another preferred embodiment, the suitably protected alpha-amino acids are selected from the group consisting of protected analogues of: pipecolic acid; 3-hydroxyleucine; isoleucine; N-methylalanine; N-methyl-3-hydroxyleucine; and leucine.

In another preferred embodiment, the suitably protected alpha-amino acids are selected from the group consisting of protected analogues of: pipecolic acid; 3-hydroxyleucine; isoleucine; N-methylalanine; N-methyl-3-hydroxyleucine; and 2-amino-4-methyl-5-hexenoic acid.

In another preferred embodiment, the suitably protected alpha-amino acids are selected from the group consisting of protected analogues of: pipecolic acid; 3-hydroxyleucine; isoleucine; N-methylalanine; N-methyl-3-hydroxyleucine; and 2-amino-4-methylhexanoic acid.

In another preferred embodiment, the suitably protected alpha-amino acids are selected from the group consisting of protected analogues of: pipecolic acid; 3-hydroxyleucine; isoleucine; N-methylalanine; N-methyl-3-hydroxyleucine; and 2-amino-4-methyl-5-hexenoic acid.

Those persons skilled in the art will appreciate that other synthetic routes may be used to synthesize the compounds of the invention. In addition, those persons skilled in the art will appreciate that, in the course of preparing the compounds of the invention, the functional groups of intermediate compounds may need to be protected by protecting groups. Functional groups which it may be desirable to protect include, but are not limited to: hydroxyl; amino; and carboxylic acid groups. Protecting groups may be added and removed in accordance with techniques that are well known to those persons skilled in the art. The use of protecting groups is described in, for example, J. W. F. McOmie (ed.), Protective Groups in Organic Chemistry, Plenum Press, London, 1973 and T. W. Greene and P. G. M. Wutz, Protective Groups in, Organic Synthesis, 2^{nd} edition, Wiley, New York, 1991.

The compounds of the invention may be further purified using techniques known to those skilled in the art. Such techniques include chromatographic methods. Liquid chromatographic methods, such as reversed-phase liquid chromatography and high performance liquid chromatography, are preferred.

Preferred purification processes are described in the Examples.

The isolation and purification methods chosen can be monitored at each step by performing in vitro and/or in vivo cytotoxicity assays as are known to those skilled in the art.

As described in the Examples, compounds within the scope of the invention have been determined to have cytotoxic properties in tests which are predictive of cytotoxic (including anti-cancer) activity in mammals, including humans.

In particular, the isolated compounds of Formula Ia; wherein R₄ is iso-propyl, R₈ is 1-hydroxy-2-methylpropyl and R₁₀ is 2-methyl-3-butenyl; R₄ is sec-butyl, R₈ is 1-hydroxy-2-methylpropyl and R₁₀ is iso-butyl; R₄ is sec-butyl, R₈ is 1-hydroxy-2-methylpropyl and R₁₀ is 2-methyl-3-butenyl; R₄ is sec-butyl, R₈ is 1-hydroxy-2-methylpropyl and R₁₀ is 2-methylbutyl; or R₄ is sec-butyl, R₈ is 1-hydroxy-2-methylbutyl and R₁₀ is 2-methyl-3-butenyl; have been evaluated against P388, a murine leukemia cell line and two human tumour cell lines—human colon cancer, HCT116; and human breast cancer, MCF7. Against P388, the compounds exhibit a range of activity of greater than two orders of magnitude, with the IC₅₀ ranging from 0.13 nM to 56 nM. Against HCT116, the compounds exhibit a range of activity from 0.3 nM to 11.6 nM, and against MCF7 from 0.9 nM to 8.3 nM.

The compounds described in the Examples are active against human cancer cell lines, such as HCT116 and MCF7, at concentrations comparable with or significantly lower than existing anticancer drugs. For example, the IC₅₀ values against HCT116 range from 0.2 ng/mL to 9.3 ng/mL for these compounds, compared with 910 ng/mL for 5-fluorouracil, 1650 ng/mL for cisplatin and 3945 ng/mL for tamoxifen. Similarly, the IC₅₀ values against MCF7 range from 0.73 ng/mL to 6.6 ng/mL for these compounds, compared with 8705 ng/mL for cisplatin and 3865 ng/mL for tamoxifen.

Initial investigation of the mode of action of the compounds described in the Examples indicate that they act by inducing apoptosis (programmed cell death) with elevation of the levels, versus control, of critical apoptotic indicators such as p53, c-myc and caspase-3. Such properties render the compounds of the invention suitable for use, alone or together with other active agents, in a number of therapeutic applications, including in anti-cancer treatments.

Advantageously, the heavily N-methylated compounds of the invention are likely to be resistant to the action of the normal range of peptidases.

Accordingly, in another aspect, the invention provides a compound of the invention for use as a medicament.

In another aspect, the present invention provides a method for the treatment or prophylaxis of cancer or another disease in a mammal comprising the step of administering a therapeutically effective amount of a compound of the invention to the mammal.

In another aspect, the present invention provides a use of a compound of the invention for the manufacture of a medicament for treating cancer or another disease.

In another aspect, the invention provides a composition comprising a compound of the invention. In a preferred embodiment, the composition is a pharmaceutical composition and further comprises a pharmaceutically acceptable carrier, diluent or excipient.

Pharmaceutically acceptable carriers, diluents and excipients are non-toxic to recipients at the dosages and concentrations employed. Each carrier, diluent and excipient must also be “acceptable” in the sense of being compatible with the other ingredients of the formulation.

The compositions of the present invention are preferably formulated for administration in unit dosage forms, such as tablets, capsules, pills, powders, granules, suppositories, sterile parenteral solutions or suspensions, sterile non-parenteral solutions or suspensions, and oral solutions or suspensions and the like, containing a therapeutically effective amount of a compound of the invention as active ingredient.

Solid or fluid unit dosage forms can be prepared for oral administration.

Powders may be prepared by comminuting the active ingredient to a suitably fine size and mixing with a similarly comminuted diluent or excipient. Suitable diluents and excipients are known to those persons skilled in the art.

Capsules may be produced by preparing a powder mixture as herein before described and filling into formed gelatine sheaths. Soft gelatine capsules may be prepared by encapsulating a slurry of active ingredients with an acceptable vegetable oil, light liquid petrolatum or other inert oil or triglyceride.

Tablets may be made by preparing a powder mixture, granulating or slugging, adding a lubricant and pressing into tablets. The powder mixture is prepared by mixing the active ingredient, suitably comminuted, with a diluent or base. Suitable diluents and bases are known to those persons skilled in the art. The powder mixture can be granulated by wetting with a binder and forcing through a screen. As an alternative to granulating, the powder mixture can be slugged, i.e. run through a tablet machine and the resulting imperfectly formed tablets broken into pieces (slugs). The slugs can be lubricated to prevent sticking to the tablet-forming dies. The lubricated mixture is then compressed into tablets.

In one embodiment, the tablet is provided with a protective coating.

Fluid unit dosage forms for oral administration, such as syrups, elixirs and suspensions, wherein a specific volume of composition contains a predetermined amount of active ingredient for administration, can be prepared. Water-soluble active ingredients can be dissolved in an aqueous vehicle together with other ingredients to form a syrup. An elixir is prepared by using a hydro-alcoholic vehicle. Suspensions can be prepared from insoluble forms in a suitable vehicle with the aid of a suspending agent.

Fluid unit dosage forms are prepared for parenteral administration utilising an active ingredient and a sterile vehicle. The active ingredient can be either suspended or dissolved in the vehicle, depending on the form and concentration used. In preparing solutions the water-soluble active ingredient can be dissolved in a suitable solvent for injection and filter sterilised before filling into a suitable vial or ampoule and sealing. Adjuvants can also be dissolved in the vehicle. Parenteral suspensions are prepared in substantially the same manner.

In addition to oral and parenteral administration, the rectal and vaginal routes may be utilised. An active ingredient can be administered by means of a suppository. A vehicle which has a melting point at about body temperature or one that is readily soluble can be utilised.

Fluid unit dosage forms for intranasal instillation are prepared utilising an active ingredient and a suitable pharmaceutical vehicle. Alternatively, a dry powder can be utilised for insufflation.

The active ingredient, together with a gaseous or liquefied propellant and suitable adjuvants as may be necessary or desirable, can be packaged into a pressurized aerosol container for use as an aerosol.

Examples of the techniques and protocols mentioned above can be found in A. R. Gennaro (ed.), Remington's Pharmaceutical Sciences, 18^{th} edition, Mack Publishing Company, Easton 1990.

The compounds and compositions of the invention may be used in combination therapies with one or more other active agents. The other active agents may form part of the same composition, or be formulated as a separate composition for administration at the same time or a different time.

Administration of the compound of Formula I or composition of the invention is preferably in a therapeutically effective amount, this being an amount sufficient to show the desired benefit to the mammal, including preventing or alleviating the symptoms of any disease or disorder being prevented or treated. The particular dosage of active ingredient to be administered will depend upon the specific disease to be treated, and various characteristics of the mammal, including age, gender, health and weight. In addition, therapeutic factors such as the site of delivery, the method of administration, any concurrent treatment, the frequency of treatment and therapeutic ratio, may also be relevant. Determining the appropriate dosage is within the ability of those persons skilled in the art.

It is expected that a useful unit dosage will comprise between about 0.1 to about 1000 mg, preferably 1 to 200 mg, of a compound of Formula I.

The following non-limiting examples are provided to illustrate the present invention and in no way limits the scope thereof.

### Examples

### Isolation

The endophytic Aspergillus sp. fungus NMI No. V08/027,588 was isolated from the root of Garcinia scortechinii, a medicinal plant of the Kuala Pilah secondary rain forest, Negeri Sembilan, Malaysia. The root was surface-sterilised, before being aseptically cut into 1 cm long segments. The flat sides of the segments were incubated on the potato dextrose agar (PDA) supplemented with chlortetracycline HCl (50 μg/ml, Sigma) and streptomycin sulphate (250 μg/ml) at 28° C. for 30 days.

### Morphological Description

On both potato dextrose (PDA) and malt agar (MA) colonies attained a diameter of 4 cm in 5 days which quickly changed from white through golden yellow to cinnamon-rufous in colour. Colonies were beige in reverse. Conidiophores upright, simple, terminating in a globose vesicle bearing phialides radiating from the entire surface; conidia (phialospores) 1 celled, globose, slightly denticulate, yellow in dry basipetal chains. Morphological features are characteristic of the genus Aspergillus. Internal transcribed spacer (ITS) sequencing revealed a 98% similarity to Aspergillus sclerotiorum (strain ATCC 16892).

### Preliminary Investigations

The extract of a small-scale NMI No. V08/027,588 culture showed excellent cytotoxicity in the P388 assay (<97.5 ng/mL). An aliquot of this crude extract was analysed (C18 HPLC), using a standard elution gradient. The chromatogram showed major peaks from between 11 min to 20 min. Bioactivity profiling showed that the activity was centred from 18-20 min. Proton NMR spectroscopy using a CapNMR probe established that a pure compound, which eluted at 18.5 min (well F6 of MT plate), showed typical peptide features. The Aspergillus sp was re-grown on a large scale to allow a full chemical investigation.

### Larger Scale Extraction

The EtOAc extract of a larger scale culture of NMI No. V08/027,588 (200 PDA plates) (2.53 g) was first partitioned between petroleum ether and MeOH. The MeOH soluble material was concentrated and fractionated on a Sephadex LH-20 column using MeOH as the solvent. Ten fractions were collected. HPLC analysis of each fraction (C18 HPLC) column established that Fr ^{#}2 contained the same peaks from the active region (18.5 min) seen in the initial analysis. The fractions containing these compounds were combined (Fr ^{#}A). Fr ^{#}A was further purified by applying a linear gradient (55-65% ACN/H₂O/0.05% formic acid/water; 20 min; analytical HPLC). From the ELSD trace, four related minor peaks were present and showed near identical UV chromophores to the original peptide. By using preparative HPLC on the analytical HPLC columns five peptides, Compounds A1, A2, A3, A4 and A5, were obtained in yields of 0.017, 0.19, 2, 0.25 and 0.12 mg respectively.

### Compound A3

The major peptide, Compound A3, was isolated as an amorphous pale yellow powder and the molecular formula, C₄₀H₆₉N₇O₉, was determined from HRESMS (MH⁺ 792.5263). The structural assignment of this initial peptide is described below in detail. Similar methods were used to assign structures to the other four peptides and these are described in lesser detail.

Two N-Me alanines were the first amino acids defined. In the first instance, the methyl group (δ_{H} 1.02, δ_{C} 15.5) was coupled to the α proton (δ_{H} 5.60, δ_{C} 50.3) (COSY and HSQC spectra). Therefore, this amino acid could be assigned as an alanine. Furthermore, a ³J_{CH} coupling from an N-Me group (δ_{H} 2.85, δ_{C} 30.0) to the α-proton was detected in the HMBC spectrum allowing assignment as an N-Me-alanine. In similar fashion, a second N-Methyl-alanine could be assigned [methyl group (δ_{H} 1.26, δ_{C} 14.3) coupled to an α-proton (δ_{H} 4.99, δ_{C} 50.7) and an N-Me group (δ_{H} 2.96, δ_{C} 31.7) with ³J_{CH} coupling to the α-proton]. The key correlations of those two amino acid units are shown in Scheme 1.

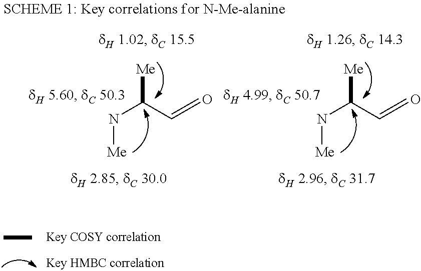

An isoleucine residue was next elucidated. An NH group (δ_{H} 8.58) was coupled to an α-proton (δ_{H} 4.47, δ_{C} 55.1) which was further coupled to a β proton (δ_{H} 1.77). However, there were two methine groups with a ¹H chemical shift of 1.77 ppm. However, the NH group also had a ³J_{CH} coupling to the β position allowing assignment as δ_{H} 1.77 and δ_{C} 33.9. Two methyl groups (δ_{H} 0.86, δ_{C} 12.3 and δ_{H} 0.87, δ_{C} 14.2) also had ⁿJ_{CH} couplings to this β position and additionally to a methylene carbon (δ_{C} 26.4). Finally, the methyl group at δ_{H} 0.86 showed H,C-coupling to the α-position. Interpretation of this data suggested this amino acid unit was an isoleucine and that the methyl group at 0.86 ppm, a doublet, was attached to the β carbon. The key correlations are shown in Scheme 2.

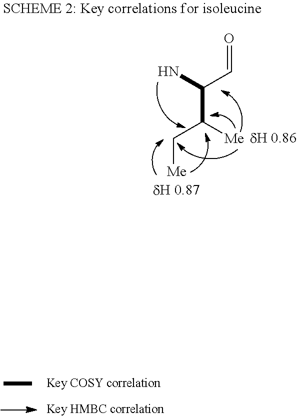

The residue with an NH group at δ_{H} 7.33 was identified as 3-hydroxyleucine. That NH proton was coupled to an α-proton (δ_{H} 4.78, δ_{C} 54.9) and further coupled to a β-proton (δ_{H} 3.43, δ_{C} 75.8) whose chemical shifts were characteristic of a carbinol system. Further structural clues came from the HMBC correlations. The α-proton also had a ³J_{CH} coupling to a methine (δ_{H} 1.77, δ_{C} 29.0). Two methyl groups (δ_{H} 0.88, δ_{C} 15.0 and δ_{H} 0.91, δ_{C} 21.3) showed correlations to the CH groups (δ_{H} 3.43, δ_{C} 75.8 and δ_{H} 1.77, δ_{C} 29.0) as well as to themselves allowing assignment of this amino acid as 3-hydroxyleucine. The key correlations are shown in Scheme 3.

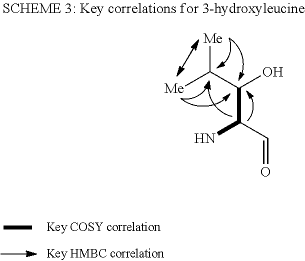

A very similar spin system was next established. This was N-Me-3-hydroxyleucine. As observed for 3-hydroxyleucine the α-proton (δ_{H} 3.79, δ_{C} 62.7) was coupled to a β-proton (δ_{H} 3.63, δ_{C} 70.4), part of a carbinol system. Two methyl groups (δ_{H} 0.76, δ_{C} 15.6 and δ_{H} 0.81, δ_{C} 21.5) showed correlations to the β- and γ-CH groups (δ_{H} 3.63, δ_{C} 70.4 and δ_{H} 1.38, δ_{C} 29.5) as well as each other in the HMBC spectrum. Furthermore, there was an N-Me group (δ_{H} 2.86, δ_{C} 29.7) with a ³J_{CH} coupling to the α-position thus defining an N-Me-3-hydroxyleucine residue (see Scheme 4).

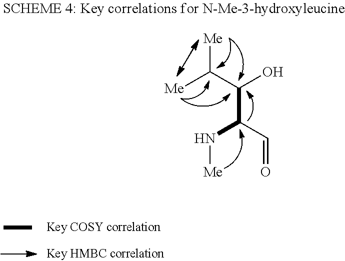

Just one NH group (δ_{H} 7.82) remained. From the COSY spectrum this NH was coupled to an α-proton (δ_{H} 4.69, δ_{C} 49.0) and then on-coupled to a methylene group (δ_{H} 1.60), which in turn was coupled to a methine (δ_{H} 2.02 δ_{C} 36.1) (see Scheme 5). Further details of the structure arose from consideration of the H,C correlations in the HMBC spectrum. The vinyl group determined from the COSY and HSQC experiments (CH δ_{H} 5.65, δ_{C} 143.0 and CH₂ δ_{H} 4.86 and 5.00, δ_{C} 115.8), had ²J_{CH} and ³J_{CH} couplings to the carbon at 36.1 ppm allowing attachment of the vinyl group to the γ-position. Furthermore, the methyl protons (δ_{H} 0.93, δ_{C} 21.3) had ³J_{CH} couplings to the β-position as well as to a vinyl group carbon (δ_{C} 143.0) fixing the position of this methyl also at the γ-position. Therefore, this amino acid unit was resolved as the rare amino acid 2-amino-4-methyl-5-hexenoic acid.

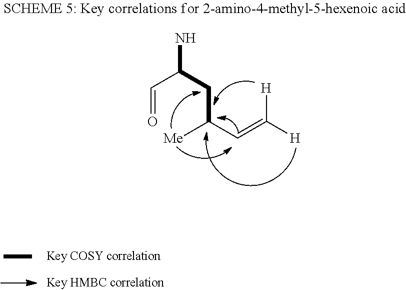

At this point only one α-proton (δ_{H} 5.00, δ_{C} 52.5) remained unassigned. From HSQC date and a consideration of molecular formula data it was ascertained there were four methylene groups left unassigned. A combination of COSY and TOCSY data, in combination with chemical shift data, were used to establish this chain of CH and CH₂ groups. The starting points were the characteristic α-proton (δ_{H} 5.00, δ_{C} 52.5) at one end of the chain and the equally distinctive CH₂ group attached to N at the other end of the chain (δ_{H} 2.88 4.02, δ_{C} 43.9). COSY gave the linkages between each methylene (see Scheme 6) and allowed assignment of the final amino acid unit as pipecolic acid.

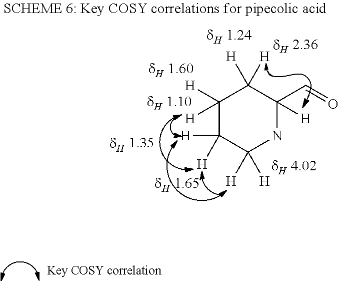

To establish the sequence of the amino acid units in the peptide ³J_{CH} couplings between the amino acid units in the HMBC experiment were utilised. But, it was found that three amino acid units had correlations to a carbonyl carbon at around 173 ppm, leaving an element of ambiguity. The more highly resolved ¹³C IMPRESS experiment was run and confirmed that the ¹³C chemical shifts at around 173 ppm came from two different amino acid with a difference of only 0.06 ppm which could not be resolved in an HMBC experiment. With the IMPRESS experiment the correlations from these two amino acids could be distinguished and assigned. The third amino acid had a carbonyl chemical shift value of 174.0 ppm and was readily resolved and also assigned. The completed peptide structure is shown below with the key H,C couplings between the amino acid units.

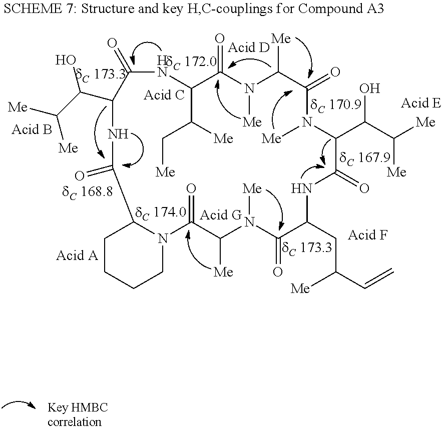

TABLE 1
NMR data for Compound A3

| Amino Acid | Position | δ_{C}, ppm | δ_{H}, ppm | COSY | HMBC |
|---|---|---|---|---|---|
| A: Pipecolic acid | 1 > CO | 168.8 | | | |
| | 2-CH | 52.5 | 5 | H-3′ | >CO of 3-hydroxyleucine |
| | 3-CH₂ | 25.9 | 1.24 | | |
| | 3′-CH₂ | 25.9 | 2.36 | H-2 | |
| | 4-CH₂ | 21.4 | 1.1 | H-5, H-5′ | |
| | 4′-CH₂ | 21.4 | 1.6 | | |
| | 5-CH₂ | 25.9 | 1.35 | H-4, H-6 | |
| | 5′-CH₂ | 25.9 | 1.65 | H-4, H-6 | |
| | 6-CH₂ | 43.9 | 2.88 | H-5, H-5′, H-6′ | |
| | 6′-CH₂ | 43.9 | 4.02 | H-6 | |
| B: 3-Hydroxy- | 1 > CO | 173.3 | | | |
| leucine | 2-CH | 54.9 | 4.78 | H-3, NH | C-3, >CO of pipecolic acid, 3- |
| | | | | | hydroxyleucine |
| | 3-CH | 75.8 | 3.43 | H-2 | |
| | 4-CH | 29 | 1.77 | | |
| | 5-Me | 15 | 0.88 | | C-3, C-4, C-6 |
| | 6-Me | 21.3 | 0.91 | | C-3, C-4, C-5 |
| | NH | | 7.33 | H-2 | >CO of pipecolic acid |
| | OH | | | | |
| C: Isoleucine | 1 > CO | 172 | | | |
| | 2-CH | 55.1 | 4.47 | H-3, NH | >CO of isoleucine |
| | 3-CH | 33.9 | 1.77 | H-2, H-6 | |
| | 4-CH₂ | 26.4 | 1.38 | | |
| | 4′-CH₂ | 26.4 | 1.48 | | |
| | 5-Me | 14.2 | 0.87 | | C-3, C-4 |
| | 6-Me | 12.3 | 0.86 | H-3 | C-2, C-3, C-4 |
| | NH | | 8.58 | H-2 | C-2, C-3, >CO of 3-hydroxy- |
| | | | | | leucine |
| D: N-Methylalanine | 1 > CO | 170.9 | | | |
| | 2-CH | 50.3 | 5.6 | H-3 | >CO of isoleucine, N- |
| | | | | | methylalanine |
| | 3-Me | 15.5 | 1.02 | H-2 | C-2, >CO of N-methylalanine |
| | N—Me | 30 | 2.85 | | C-2, >CO of isoleucine, |
| E: N-Methyl-3- | 1 > CO | 167.9 | | | |
| hydroxyleucine | 2-CH | 62.7 | 3.79 | H-3 | C-3, N—Me, >CO of N- |
| | | | | | methylalanine, N-methyl-3- |
| | | | | | hydroxyleucine |
| | 3-CH | 70.4 | 3.63 | H-2 | C-5, C-6 |
| | 4-CH | 29.5 | 1.38 | | |
| | 5-Me | 15.6 | 0.76 | | C-3, C-4, C-6 |
| | 6-Me | 21.5 | 0.81 | | C-3, C-4, C-5 |
| | N—Me | 29.7 | 2.86 | | |
| | OH | | | | |
| F: 2-Amino-4- | 1 > CO | 173.3 | | | |
| methyl-5-hexenoic | 2-CH | 49 | 4.69 | H-3′, NH | |
| acid | 3-CH₂ | 36.2 | 1.5 | | |
| | 3′-CH₂ | 36.2 | 1.6 | H-2, H-4 | |
| | 4-CH | 36 | 2.02 | H-3′ | |
| | 5-CH | 143 | 5.65 | | C-4 |
| | 6-CH₂ | 115.8 | 4.86 | | C-4 |
| | 6′-CH₂ | 115.8 | 5 | | C-4 |
| | 7-Me | 21.4 | 0.93 | | C-3, C-5 |
| | NH | | 7.82 | H-2 | >CO of N-methyl-3- |
| | | | | | hydroxyleucine |
| G: N-Methylalanine | 1 > CO | 174 | | | |
| | 2-CH | 50.7 | 4.99 | H-3 | >CO of N-methylalanine |
| | 3-Me | 14.3 | 1.26 | H-2 | C-2, >CO of N-methylalanine |
| | N—Me | 31.7 | 2.96 | | C-2, >CO of 2-amino-4- |
| | | | | | methyl-5-hexenoic acid |

### Compound A4

Compound A4 was obtained as a pale yellow powder with a molecular formula C₄₀H₇₁N₇O₉ which was established on the basis of HRESI mass spectrometry (MH⁺ 794.5357). This corresponds to two protons more than Compound A3. In the ¹H and HSQC spectra there were no signals corresponding to olefinic protons and carbons. This suggested that the difference between Compound A3 and Compound A4 was that the vinyl group of 2-amino-4-methyl-5-hexenoic acid (amino acid F) had been reduced. Careful analysis of the COSY and TOCSY spectra revealed that the α-proton (δ_{H} 4.83, δ_{C} 48.8) had correlations to a methylene group (δ_{H}1.47, δ_{C} 36.1) and was on-coupled to a methine group (δ_{H}1.78, δ_{C} 33.5). And, from the HSQC and HMBC spectra, the doublet methyl group (δ_{H} 0.76, δ_{C} 19.8) had H,C-couplings to the same methylene group (δ_{H}1.47, δ_{C} 36.1) and methine group (δ_{H} 1.78, δ_{C} 33.5), establishing the relationship this methyl group had with the COSY-defined spin system. Another new methyl group, a triplet (δ_{H} 0.75, δ_{C} 11.8) also had a ³J_{CH} coupling to the methine group at 1.78 ppm as well as a ²J_{CH} coupling to an alternative methylene group (δ_{H} 0.96, 1.14, δ_{C} 27.5) establishing the structure of the new amino acid as 2-amino-4-methyl-hexanoic acid.

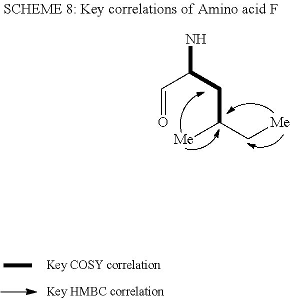

TABLE 2
¹³C and ¹H data comparison of acid F from Compound A3
and Compound A4

| | Cmpd A3 | | | Cmpd A4 | | |
|---|---|---|---|---|---|---|
| Acid F | Position | δ_{C}, ppm | δ_{H}, ppm | Position | δ_{C}, ppm | δ_{H}, ppm |
| | 1 > CO | 172.8 | | 1 > CO | 173.4 | |
| | 2-CH | 49.0 | 4.69 | 2-CH | 48.8 | 4.83 |
| | 3-CH₂ | 36.2 | 1.50, 1.60 | 3-CH₂ | 36.1 | 1.47 |
| | 4-CH | 36.0 | 2.02 | 4-CH | 33.5 | 1.78 |
| | 5-CH | 143.0 | 5.65 | 5-CH₂ | 27.5 | 0.96, 1.14 |
| | 6-CH₂ | 115.8 | 4.86, 5.00 | 6-CH₃ | 11.8 | 0.75 |
| | 7-CH₃ | 21.4 | 0.93 | 7-CH₃ | 19.8 | 0.76 |
| | NH | | 7.82 | NH | | 7.78 |

Signals in other parts of the molecule, including all NH and N-Me groups, were the same as Compound A3. The structure of Compound A4 is shown in Scheme 9.

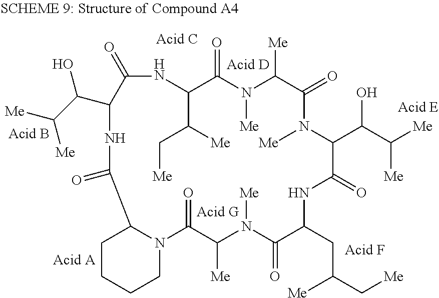

### Compound A1

Compound A1, the first compound to elute from the HPLC separation, was the next structure assigned. It too was isolated as a pale yellow powder and had a molecular formula of C₃₉H₆₇N₇O₉ as determined by HREIMS (MH⁺ 778.5051). This corresponds to one less methylene group when compared to Compound A3. By carefully comparing the HSQC spectrum of Compound A1 to that of Compound A3 it was observed that the chemical shift of the α-proton in acid C (isoleucine) had changed from 4.45 ppm in Compound A3 to 4.38 ppm in Compound A1. That α-proton showed H—H coupling to a methine proton (δ_{H} 2.02, δ_{C} 28.2) which was also different from the proton at this position in Compound A3 (δ_{H}1.77, δ_{C} 33.9). In the HSQC spectra correlations for an isoleucine methylene group and two methyl singles (δ_{H} 0.86 and 0.86) were replaced by two other methyl signals (δ_{H} 0.92 and 1.03). In the HMBC spectra these two new methyl groups showed typical valine H,C-couplings to α-position (δ_{C} 57.6) and to the β-position (δ_{C} 28.2), as well as to each other, confirming that in Compound A1 the isoleucine at amino acid C had been substituted with a valine. Key correlations are shown below. This substitution was in keeping with the observed molecular formula for Compound A1. No other major changes in the NMR spectra were discernible.

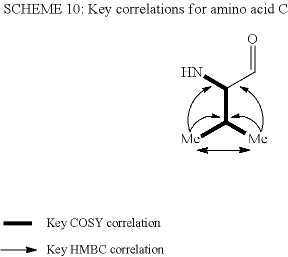

TABLE 3
¹³C and ¹H data comparison of acid C from Compound A1
and Compound A3

| | Cmpd A1 | | | Cmpd A3 | | |
|---|---|---|---|---|---|---|
| Acid C | Position | δ_{C}, ppm | δ_{H}, ppm | Position | δ_{C}, ppm | δ_{H}, ppm |
| | 1 > CO | 172.5 | | 1 > CO | 172.0 | |
| | 2-CH | 57.6 | 4.38 | 2-CH | 55.1 | 4.47 |
| | 3-CH | 28.2 | 2.02 | 3-CH | 33.9 | 1.77 |
| | | | | 4-CH₂ | 26.4 | 1.38, 1.48 |
| | 4-CH₃ | 17.5 | 0.92 | 5-CH₃ | 14.2 | 0.86 |
| | 5-CH₃ | 19.9 | 1.03 | 6-CH₃ | 12.3 | 0.86 |
| | NH | | 8.65 | NH | | 8.58 |

Other signals of Compound A1 remained similar as in Compound A3, and all the NH and N-Me remained at the same chemical shifts. Therefore, the structure of Compound A1 is shown in Scheme 11.

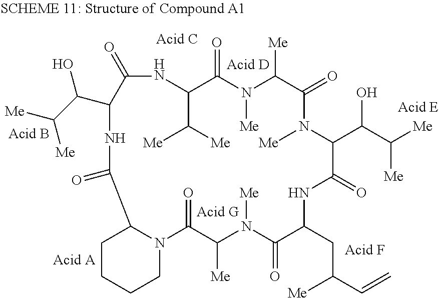

### Compound A2

Compound A2 was obtained as a pale yellow powder and has the molecular formula C₃₉H₆₉N₇O₉ by HREIMS (MH⁺ 780.5214). Compound A2 nominally has two more hydrogen atoms than Compound A1. The initial assumption was that Compound A2 was related to Compound A1 simply by hydrogenation of the vinyl group (there were no olefinic protons or carbons discernible) as had been observed for Compound A3/Compound A4. However, from a careful assignment of all the NMR data it was apparent that the amino acid C was identical to that in Compound A3 and Compound A4. That is, isoleucine rather than valine and that the major difference was centred on amino acid F. As noted there were no olefinic signals present in the spectra associated with Compound A2, but the residue at F could not be 2-amino-4-methylhexanoic acid based on MF arguments and the NMR data. There were signals for two doublet methyl groups in amino acid F (δ_{H} 0.84 and δ_{H} 0.86) replacing the methyl groups (doublet and triplet at δ_{H} 0.75 and δ_{H} 0.76) found in 2-amino-4-methylhexanoic acid. Analysis of the HMBC spectrum established that these two double methyl groups were part of a leucine system (H,C couplings to C-3 (δ_{C} 38.0) and C-4 (δ_{C} 25.72)).

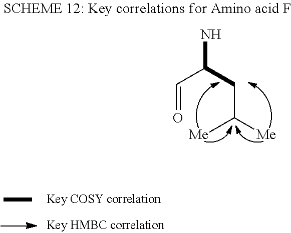

TABLE 4
¹³C and ¹H data comparison of acid F from Compound A2
and Compound A4

| | Cmpd A2 | | | Cmpd A4 | | |
|---|---|---|---|---|---|---|
| Acid F | Position | δ_{C}, ppm | δ_{H}, ppm | Position | δ_{C}, ppm | δ_{H}, ppm |
| | 1-Carbonyl | 173.2 | | 1-Carbonyl | 173.4 | |
| | 2-CH | 49.1 | 4.84 | 2-CH | 48.8 | 4.83 |
| | 3-CH₂ | 38.0 | 1.38, 1.56 | 3-CH₂ | 36.1 | 1.47 |
| | 4-CH | 25.7 | 1.54 | 4-CH | 33.5 | 1.78 |
| | | | | 5-CH₂ | 27.5 | 0.96, 1.14 |
| | 5-CH₃ | 23.8 | 0.84 | 6-CH₃ | 11.8 | 0.75 |
| | 6-CH₃ | 21.5 | 0.86 | 7-CH₃ | 19.8 | 0.76 |
| | NH | | 7.82 | NH | | 7.78 |

All other amino acids are the same as in Compound A3, so the structure of Compound A2 is as shown in Scheme 13.

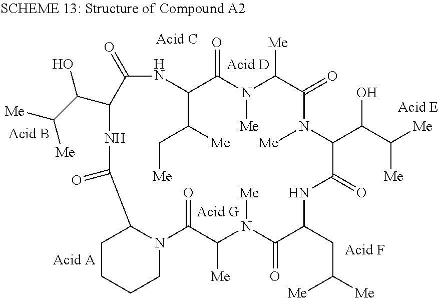

### Compound A5

Compound A5 was isolated as a pale yellow powder and the HREIMS (MH⁺ 806.5383) suggested the molecular formula C₄₁N₇₁N₇O₉. This represents an additional CH, more than that observed for Compound A3. Analysis of all NMR data and assignment of structure indicated a close comparison with Compound A3, except for amino acid E. The amino acid residue found in Compound A3 at amino acid E was N-methyl-3-hydroxyleucine. In Compound A5 the two methyl groups of N-methyl-3-hydroxyleucine (5-CH₃ δ_{H} 0.76, δ_{C} 15.6 and 6-CH₃ δ_{H} 0.86, δ_{c} 29.7) have been replaced in the HSQC spectra by two alternative methyl groups (δ_{H} 0.75, δ_{C} 12.4 and δ_{H}0.77, δ_{c} 13.5). From the TOCSY spectrum the methyl at δ_{H} 0.75, δ_{C} 12.4 has correlations to methylene (δ_{H}1.11 and 1.27) and methine (δ_{H}1.10) groups. This evidence suggested that the amino acid residue has changed from N-methyl-3-hydroxyleucine to a new amino acid N-methyl-2-amino-3-hydroxy-4-methylhexanoic acid. The HMBC data were able to confirm this. For example, the 4-methyl group has a ³J_{CH} coupling to the β carbon (δ_{C} 68.0) placing it at the 4-position rather than the 6-position. The key TOCSY and HMBC correlations observed are shown in Scheme 14.

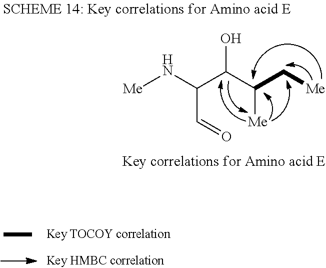

TABLE 5
¹³C and ¹H data comparison of acid E from Compound A5
and Compound A3

| | Cmpd A5 | | | Cmpd A3 | | |
|---|---|---|---|---|---|---|
| Acid E | Position | δ_{C}, ppm | δ_{H}, ppm | Position | δ_{C}, ppm | δ_{H}, ppm |
| | 1 > CO | 167.9 | | 1 > CO | 170.9 | |
| | 2-CH | 63.1 | 3.86 | 2-CH | 62.7 | 3.79 |
| | 3-CH | 68.0 | 3.79 | 3-CH | 70.4 | 3.63 |
| | 4-CH | 35.9 | 1.1 | 4-CH | 29.5 | 1.38 |
| | 5-CH₂ | 27.9 | 1.11, 1.27 | | | |
| | 6-CH₃ | 12.4 | 0.75 | 5-CH₃ | 15.6 | 0.76 |
| | 7-CH₃ | 13.5 | 0.77 | 6-CH₃ | 21.5 | 0.81 |
| | N—Me | 29.8 | 2.87 | N—Me | 29.7 | 2.86 |
| | OH | | 4.96 | OH | | |

All other amino acids remained the same as in the Compound A3, therefore, the structure of Compound A5 is as shown in Scheme 15.

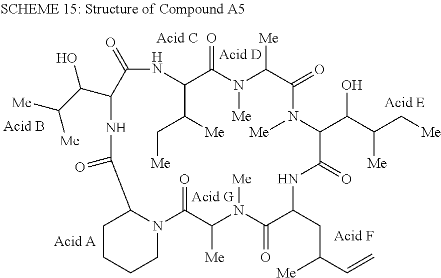

The structures of all of the Compounds A1-A5 are shown in FIG. 1, while a complete listing of the NMR data for each compound appears in the following Tables.

TABLE 6
NMR data for Compound A1

| Amino Acids | Position | δ_{C}, | δ_{H}, | COSY | HMBC |
|---|---|---|---|---|---|
| A: pipecolic acid | 1-CH | 52.5 | 5 | | |
| | 2-CH₂ | 25.9 | 1.25 | | |
| | | 25.9 | 2.37 | | |
| | 3-CH₂ | 21.5 | 1.1 | | |
| | | 21.5 | 1.61 | | |
| | 4-CH₂ | 25.9 | 1.34 | | |
| | | 25.9 | 1.63 | | |
| | 5-CH₂ | 43.9 | 2.88 | | |
| | | 43.9 | 4.04 | | |
| | Carbonyl | 168.7 | | | |
| B: 3-hydroxyleucine | 1-CH | 55.1 | 4.78 | H-3, NH | C-3, >CO of B |
| | 2-CH | 75.7 | 3.46 | H-2, OH | |
| | 3-CH | 29 | 1.79 | | |
| | 4-Me | 15.2 | 0.88 | | C-3, C-4, C-6 |
| | 5-Me | 21.4 | 0.91 | | C-3, C-4, C-5 |
| | NH | | 7.39 | H-2 | >CO of A |
| | OH | | 4.77 | H-3 | |
| | Carbonyl | 173.4 | | | |
| C: valine | >CO | 172.5 | | | |
| | 2-CH | 57.6 | 4.38 | H-3, NH | C-3, >CO of C |
| | 3-CH | 28.2 | 2.02 | H-2, H-4, H-5 | |
| | 4-Me | 17.5 | 0.92 | H-3 | C-2, C-3, C-5 |
| | 5-Me | 19.9 | 1.03 | H-3 | C-2, C-3, C-4 |
| | NH | | 8.65 | H-2 | >CO of B |
| D: N-methylalanine | >CO | 171.8 | | | |
| | 2-CH | 50.3 | 5.58 | H-3 | C-3 |
| | 3-Me | 15.6 | 1.02 | H-2 | C-2, >CO of D |
| | N—Me | 30.3 | 2.86 | | C-2, >CO of C |
| E: N-methyl-3-hydroxyleucine | >CO | 168 | | | |
| | 2-CH | 63.1 | 3.78 | H-3 | >CO of E |
| | 3-CH | 70.4 | 3.63 | H-2, OH | |
| | 4-CH | 29.5 | 1.37 | | |
| | 5-Me | 15.8 | 0.77 | | C-3, C-4, C-6 |
| | 6-Me | 21.7 | 0.82 | | C-3, C-4, C-5 |
| | N—Me | 29.7 | 2.85 | | C-2, >CO of D |
| | OH | | 5.05 | H-3 | C-4 |
| F: 2-amino-4-methyl-5-hexenoic | >CO | 173.4 | | | |
| acid | 2-CH | 49 | 4.71 | H-3, NH | |
| | 3-CH₂ | 36.5 | 1.51 | H-2 | |
| | | 36.5 | 1.59 | | |
| | 4-CH | 36.2 | 2.01 | | |
| | 5-CH | 143.1 | 5.66 | | |
| | 6-CH₂ | 115.7 | 4.87 | | C-4 |
| | | 115.7 | 5 | | C-4 |
| | 7-Me | 21.4 | 0.95 | | C-3, C-5 |
| | NH | | 7.83 | H-2 | >CO of E |
| G: N-methylalanine | >CO | 174.3 | | | |
| | 2-CH | 51.2 | 4.99 | H-3 | C-3, >CO of G |
| | 3-Me | 14.3 | 1.26 | H-2 | C-2, >CO of G |
| | N—Me | 31.7 | 2.96 | | C-2, >CO of F |

TABLE 7
NMR data for Compound A2

| Amino Acids | Position | δ_{C}, ppm | δ_{H}, ppm | COSY | HMBC |
|---|---|---|---|---|---|
| A: pipecolic acid | >CO | 168.7 | | | |
| | 2-CH | 55.4 | 5 | | |
| | 3-CH₂ | 25.9 | 1.26 | | |
| | | 25.9 | 2.38 | | |
| | 4-CH₂ | 21.5 | 1.1 | | |
| | | 21.5 | 1.62 | | |
| | 5-CH₂ | 25.9 | 1.37 | | |
| | | 25.9 | 1.67 | | |
| | 6-CH₂ | 43.8 | 2.91 | | |
| | | 43.8 | 4.06 | | |
| B: 3-hydroxyleucine | >CO | 173.4 | | | |
| | 2-CH | 55.1 | 4.79 | H-3, NH | C-3, >CO of A, B |
| | 3-CH | 75.7 | 3.46 | H-2 | |
| | 4-CH | 29 | 1.79 | | |
| | 5-Me | 15.2 | 0.89 | | C-3, C-4, C-6 |
| | 6-Me | 21.4 | 0.92 | | C-3, C-4, C-5 |
| | NH | | 7.39 | H-2 | >CO of A |
| | OH | | | | |
| C: isoleucine | >CO | 172.3 | | | |
| | 2-CH | 55.6 | 4.47 | H-3, NH | C-3, >CO of C |
| | 3-CH | 33.8 | 1.77 | H-2 | |
| | 4-CH2 | 26.2 | 1.39 | | |
| | | 16.2 | 1.5 | | |
| | 5-Me | 14.2 | 0.88 | | C-3 |
| | 6-Me | 12.4 | 0.88 | | C-2, C-3 |
| | NH | | 8.55 | H-2 | C-2, C-3, >CO of B |
| D: N-methylalanine | >CO | 171.3 | | | |
| | 2-CH | 50 | 5.6 | H-3 | C-3, N—Me, >CO of D |
| | 3-Me | 15.5 | 1.01 | H-2 | C-2, >CO of D |
| | N—Me | 30 | 2.83 | | C-2, >CO of C |
| E: N-methyl-3-hydroxyleucine | >CO | 168 | | | |
| | 2-CH | 63.1 | 3.81 | H-3 | C-3, >CO of E |
| | 3-CH | 70.5 | 3.63 | H-2, OH | |
| | 4-CH | 29.4 | 1.39 | | |
| | 5-Me | 15.6 | 0.78 | | C-3, C-6 |
| | 6-Me | 21.7 | 0.83 | | C-3, C-5 |
| | N—Me | 29.7 | 2.86 | | C-2, >CO of D |
| | OH | | 5.02 | H-3 | C-2, C-3, C-4 |
| F: leucine | >CO | 173.2 | | | |
| | 2-CH | 49.1 | 4.84 | H-3, NH | |
| | 3-CH₂ | 38 | 1.38 | H-2 | |
| | | 38 | 1.56 | | |
| | 4-CH | 25.7 | 1.54 | | |
| | 5-Me | 23.8 | 0.84 | | C-3, C-4, C-6 |
| | 6-Me | 21.5 | 0.86 | | C-3, C-4, C-5 |
| | NH | | 7.82 | H-2 | >CO of E |
| G: N-Methylalanine | >CO | 174.3 | | | |
| | 2-CH | 51.2 | 5 | H-3 | C-3, >CO of G |
| | 3-Me | 14.3 | 1.27 | H-2 | C-2, >CO of G |
| | N—Me | 31.8 | 3.04 | | C-2, >CO of F |

TABLE 8
NMR data for Compound A3

| Amino Acids | Position | δ_{C}, ppm | δ_{H}, ppm | COSY | HMBC |
|---|---|---|---|---|---|
| A: pipecolic acid | >CO | 168.8 | | | |
| | 2-CH | 52.5 | 5 | H-3′ | >CO of B |
| | 3-CH₂ | 25.9 | 1.24 | | |
| | 3′-CH₂ | 25.9 | 2.36 | H-2 | |
| | 4-CH₂ | 21.4 | 1.1 | H-5, H-5′ | |
| | 4′-CH₂ | 21.4 | 1.6 | | |
| | 5-CH₂ | 25.9 | 1.35 | H-4, H-6 | |
| | 5′-CH₂ | 25.9 | 1.65 | H-4, H-6 | |
| | 6-CH₂ | 43.9 | 2.88 | H-5, H-5′, H-6′ | |
| | 6′-CH₂ | 43.9 | 4.02 | H-6 | |
| B: 3-hydroxyleucine | >CO | 173.3 | | | |
| | 2-CH | 54.9 | 4.78 | H-3, NH | C-3, >CO of A, B |
| | 3-CH | 75.8 | 3.43 | H-2 | |
| | 4-CH | 29 | 1.77 | | |
| | 5-Me | 15 | 0.88 | | C-3, C-4, C-6 |
| | 6-Me | 21.3 | 0.91 | | C-3, C-4, C-5 |
| | NH | | 7.33 | H-2 | >CO of A |
| | OH | | | | |
| C: isoleucine | >CO | 172 | | | |
| | 2-CH | 55.1 | 4.47 | H-3, NH | >CO of C |
| | 3-CH | 33.9 | 1.77 | H-2, H-6 | |
| | 4-CH2 | 26.4 | 1.38 | | |
| | 4′-CH2 | 26.4 | 1.48 | | |
| | 5-Me | 14.2 | 0.87 | | C-3, C-4 |
| | 6-Me | 12.3 | 0.86 | H-3 | C-2, C-3, C-4 |
| | NH | | 8.58 | H-2 | C-2, C-3, >CO of |
| | | | | | B |
| D: N-methylalanine | >CO | 170.9 | | | |
| | 2-CH | 50.3 | 5.6 | H-3 | >CO of C, D |
| | 3-Me | 15.5 | 1.02 | H-2 | C-2, >CO of D |
| | N—Me | 30 | 2.85 | | C-2, >CO of C |
| E: N-methyl-3-hydroxyleucine | >CO | 167.9 | | | |
| | 2-CH | 62.7 | 3.79 | H-3 | C-3, N—Me, >CO |
| | | | | | of D, E |
| | 3-CH | 70.4 | 3.63 | H-2 | C-5, C-6 |
| | 4-CH | 29.5 | 1.38 | | |
| | 5-Me | 15.6 | 0.76 | | C-3, C-4, C-6 |
| | 6-Me | 21.5 | 0.81 | | C-3, C-4, C-5 |
| | N—Me | 29.7 | 2.86 | | |
| | OH | | | | |
| F: 2-amino-4-methyl-5-hexenoic | >CO | 173.3 | | | |
| acid | 2-CH | 49 | 4.69 | H-3′, NH | |
| | 3-CH₂ | 36.2 | 1.5 | | |
| | 3′-CH₂ | 36.2 | 1.6 | H-2, H-4 | |
| | 4-CH | 36 | 2.02 | H-3′ | |
| | 5-CH | 143 | 5.65 | | C-4 |
| | 6-CH₂ | 115.8 | 4.86 | | C-4 |
| | 6′-CH₂ | 115.8 | 5 | | C-4 |
| | 7-Me | 21.4 | 0.93 | | C-3, C-5 |
| | NH | | 7.82 | H-2 | >CO of E |
| G: N-methylalanine | >CO | 174 | | | |
| | 2-CH | 50.7 | 4.99 | H-3 | >CO of G |
| | 3-Me | 14.3 | 1.26 | H-2 | C-2, >CO of G |
| | N—Me | 31.7 | 2.96 | | C-2, >CO of F |

TABLE 9
NMR data for Compound A4

| Amino Acids | Position | δ_{C}, ppm | δ_{H}, ppm | COSY | HMBC |
|---|---|---|---|---|---|
| A: pipecolic acid | >CO | 168.9 | | | |
| | 2-CH | 52.6 | 4.97 | H-3, H-3′ | >CO of A |
| | 3-CH₂ | 25.8 | 1.24 | H-2 | |
| | | 25.8 | 2.34 | H-2 | |
| | 4-CH₂ | 21.4 | 1.09 | | |
| | | 21.4 | 1.61 | | |
| | 5-CH₂ | 25.8 | 1.34 | H-6, H-6′ | |
| | | 25.8 | 1.63 | H-6, H-6′ | |
| | 6-CH₂ | 43.8 | 2.98 | H-5, H-5′ | |
| | | 43.8 | 4.02 | H-5, H-5′ | |
| B: 3-hydroxy-leucine | >CO | 173.4 | | | |
| | 2-CH | 55 | 4.75 | H-3, NH | >CO of B |
| | 3-CH | 75.7 | 3.44 | H-2, OH | |
| | 4-CH | 29 | 1.77 | | |
| | 5-Me | 15 | 0.84 | | C-3, C-4, C-6 |
| | 6-Me | 21.1 | 0.89 | | C-3, C-4, C-5 |
| | NH | | 7.43 | H-2 | >CO of A |
| | OH | | 5.12 | H-3 | |
| C: isoleucine | >CO | 172.4 | | | |
| | 2-CH | 55.5 | 4.45 | H-3, NH | C-3, >CO of C |
| | 3-CH | 33.6 | 1.76 | H-2 | |
| | 4-CH2 | 27.5 | 1.39 | | |
| | 5-Me | 12.1 | 0.84 | | C-4 |
| | 6-Me | 14.1 | 0.83 | | C-2, C-3, C-4 |
| | NH | | 8.65 | H-2 | >CO of B |
| D: N-methyl-alanine | >CO | 171.4 | | | |
| | 2-CH | 50 | 5.6 | H-3 | >CO of D |
| | 3-Me | 15.3 | 1 | H-2 | C-2, >CO of D |
| | N—Me | 29.8 | 2.8 | | C-2, >CO of C |
| E: N-methyl-3-hydroxy-leucine | >CO | 168.2 | | | |
| | 2-CH | 63.2 | 3.8 | H-3 | C-3, C-4, >CO of E |
| | 3-CH | 70.4 | 3.62 | H-2 | |
| | 4-CH | 29.4 | 1.35 | | |
| | 5-Me | 15.5 | 0.74 | | C-3, C-6 |
| | 6-Me | 21.4 | 0.8 | | C-3, C-5 |
| | N—Me | 29.8 | 2.81 | | C-2, >CO of D |
| | OH | | | | C-4 |
| F: 2-amino-4-methylhexanoic acid | >CO | 173.4 | | | |
| | 2-CH | 48.8 | 4.83 | H-3, NH | >CO of F |
| | 3-CH₂ | 36.1 | 1.47 | H-2, H-4 | |
| | 4-CH | 33.5 | 1.78 | H-3 | |
| | 5-CH₂ | 27.5 | 0.96 | | |
| | | 27.5 | 1.14 | | |
| | 6-Me | 11.8 | 0.75 | | C-4, C-5 |
| | 7-Me | 19.8 | 0.76 | | C-3, C-4, C-5 |
| | NH | | 7.78 | H-2 | >CO of E |
| G: N-methyl-alanine | >CO | 174.6 | | | |
| | 2-CH | 51.4 | 4.93 | H-3 | |
| | 3-Me | 14.1 | 1.25 | H-2 | C-2, >CO of G |
| | N—Me | 31.6 | 3 | | >CO of F |

TABLE 10
NMR data for Compound A5

| Amino Acids | Position | δ_{C}, ppm | δ_{H}, ppm | COSY | HMBC |
|---|---|---|---|---|---|
| A: pipecolic acid | >CO | 168.7 | | | |
| | 2-CH | 52.5 | 4.99 | | |
| | 3-CH₂ | 25.9 | 1.25 | | |
| | | 25.9 | 2.36 | | |
| | 4-CH₂ | 21.5 | 1.1 | | |
| | | 21.5 | 1.6 | | |
| | 5-CH₂ | 25.9 | 1.37 | | |
| | | 25.9 | 1.65 | | |
| | 6-CH₂ | 43.8 | 2.9 | | |
| | | 43.8 | 4.06 | | |
| B: 3-hydroxy-leucine | >CO | 173.2 | | | |
| | 2-CH | 55 | 4.78 | H-3, NH | C-3, >CO of A, B |
| | 3-CH | 75.7 | 3.41 | H-2, H-4 | |
| | | | | OH | |
| | 4-CH | 29 | 1.78 | H-3 | |
| | 5-Me | 15.2 | 0.88 | | C-3, C-4, C-6 |
| | 6-Me | 21.5 | 0.92 | | C-3, C-4, C-5 |
| | NH | | 7.34 | H-2 | >CO of A |
| | OH | | 4.93 | H-3 | C-4 |
| C: isoleucine | >CO | 172.4 | | | |
| | 2-CH | 55.7 | 4.47 | H-3, NH | >CO of C |
| | 3-CH | 33.8 | 1.76 | H-2 | |
| | 4-CH₂ | 26.4 | 1.37, 1.49 | | |
| | 5-Me | 14.2 | 0.88 | | C-3, C-4 |
| | 6-Me | 12.4 | 0.86 | | C-3, C-4 |
| | NH | | 8.59 | H-2 | >CO of B |
| D: N-methyl-alanine | >CO | 171.4 | | | |
| | 2-CH | 50.1 | 5.63 | H-3 | C-3, N—Me, >CO |
| | | | | | of D |
| | 3-Me | 15.7 | 1.02 | H-2 | C-2, >CO of D |
| | N—Me | 30 | 2.85 | | C-2, >CO of C |
| E: N-methyl-3-hydroxy-leucine | >CO | 167.9 | | | |
| | 2-CH | 63.1 | 3.86 | H-3 | C-3, N—Me, >CO |
| | | | | | of D, E |
| | 3-CH | 68 | 3.79 | H-2, OH | C-2, C-7 |
| | 4-CH | 35.9 | 1.1 | | |
| | 5-CH₂ | 27.9 | 1.11 | | |
| | | 27.9 | 1.27 | | |
| | 6-Me | 12.4 | 0.75 | | C-4, C-5 |
| | 7-Me | 13.5 | 0.77 | | C-3, C-4, C-5 |
| | N—Me | 29.8 | 2.87 | | C-2, >CO of D |
| | OH | | 4.96 | H-3 | |
| F: 2-amino-4-methyl-5-hexenoic acid | >CO | 173.2 | | | |
| | 2-CH | 49 | 4.69 | H-3, NH | |
| | 3-CH₂ | 36.4 | 1.49 | H-2 | |
| | | 36.4 | 1.61 | | |
| | 4-CH | 36 | 2 | | |
| | 5-CH | 143.1 | 5.66 | | |
| | 6-CH₂ | 115.8 | 4.86 | | C-4 |
| | | 115.8 | 5 | | C-4 |
| | 7-Me | 21.5 | 0.93 | | C-4, C-5 |
| | NH | | 7.76 | H-2 | >CO of E |
| G: N-methyl-alanine | >CO | 174.4 | | | |
| | 2-CH | 51.2 | 4.99 | H-3 | C-3, >CO of G |
| | 3-Me | 14.3 | 1.26 | H-2 | C-2, >CO of G |
| | N—Me | 31.8 | 2.96 | | C-2, >CO of F |

TABLE 11
Comparative NMR data for the Compounds A1-A5

| Amino Acids | | Cmpd A1 | Cmpd A2 | Cmpd A3 | Cmpd A4 | Cmpd5 |
|---|---|---|---|---|---|---|
| A: pipecolic | >CO | 168.65 | 168.66 | 168.8 | 168.94 | 168.67 |
| acid | 2-CH | 5.00 52.51 | 5.00 55.44 | 5.0 52.5 | 4.97 52.59 | 4.99 52.46 |
| | 3-CH₂ | 1.25 25.85 | 1.26 25.9 | 1.24 25.9 | 1.24 25.76 | 1.25 25.89 |
| | | 2.37 25.85 | 2.38 25.9 | 2.36 25.9 | 2.34 25.76 | 2.36 25.89 |
| | 4-CH₂ | 1.10 21.49 | 1.10 21.5 | 1.10 21.4 | 1.09 21.44 | 1.10 21.50 |
| | | 1.61 21.49 | 1.62 21.5 | 1.60 21.4 | 1.58 21.44 | 1.60 21.50 |
| | 5-CH₂ | 1.34 25.851 | 1.37 25.9 | 1.35 25.9 | 1.34 25.76 | 1.37 25.89 |
| | | 1.63 25.85 | 1.67 25.9 | 1.65 25.9 | 1.63 25.76 | 1.65 25.89 |
| | 6-CH₂ | 2.88 43.86 | 2.91 43.83 | 2.88 43.9 | 2.98 43.79 | 2.90 43.80 |
| | | 4.04 43.86 | 4.06 43.83 | 4.02 43.9 | 4.02 43.79 | 4.06 43.80 |
| B: 3-hydroxy- | >CO | 173.39 | 173.24 | 173.3 | 173.35 | 173.23 |
| leucine | 2-CH | 4.78 55.11 | 4.79 55.05 | 4.78 54.9 | 4.75 55.04 | 4.78 55.02 |
| | 3-CH | 3.46 75.66 | 3.46 75.70 | 3.43 75.8 | 3.44 75.68 | 3.41 75.67 |
| | 4-CH | 1.79 29.02 | 1.79 29.02 | 1.77 29.0 | 1.77 29.01 | 1.78 29.03 |
| | 5-Me | 0.88 15.18 | 0.89 15.17 | 0.88 15.0 | 0.84 14.99 | 0.88 15.23 |
| | 6-Me | 0.91 21.43 | 0.92 21.38 | 0.91 21.3 | 0.89 21.10 | 0.92 21.45 |
| | NH | 7.39 | 7.39 | 7.33 | 7.43 | 7.43 |
| | OH | 4.77 | | | 5.12 | 4.93 |
| C: A1 valine | >CO | 172.54 | 172.33 | 172 | 172.44 | 172.37 |
| A2-A5 | 2-CH | 4.38 57.61 | 4.47 55.60 | 4.47 55.1 | 4.45 55.54 | 4.47 55.65 |
| isoleucine | 3-CH | 2.02 28.15 | 1.77 33.78 | 1.77 33.9 | 1.76 33.57 | 1.76 33.83 |
| | 4-CH₂ | | 1.39 26.2 | 1.38 26.4 | 1.39 27.53 | 1.37 26.39 |
| | | | 1.50 26.2 | 1.48 26.4 | | 1.49 26.39 |
| | 5-Me | 0.92 17.48 | 0.88 14.2 | 0.86 14.2 | 0.84 12.14 | 0.86 12.39 |
| | 6-Me | 1.03 19.87 | 0.88 12.4 | 0.86 12.3 | 0.83 14.07 | 0.88 14.17 |
| | NH | 8.65 | 8.55 | 8.58 | 8.44 | 8.59 |
| D: N-methyl- | >CO | 171.28 | 171.28 | 170.9 | 171.35 | 171.38 |
| alanine | 2-CH | 5.58 50.32 | 5.60 50.03 | 5.60 50.3 | 5.60 50.03 | 5.63 50.10 |
| | 3-Me | 1.02 15.61 | 1.01 15.54 | 1.02 15.5 | 1.00 15.30 | 1.02 15.67 |
| | N-Me | 2.86 30.33 | | 2.85 30.0 | 2.80 29.81 | 2.85 30.03 |
| E: A1-A4 N- | >CO | 167.97 | 167.9 | 170.92 | 168.23 | 167.85 |
| methyl-3- | 2-CH | 3.78 63.06 | 3.81 63.14 | 3.79 62.7 | 3.80 63.17 | 3.86 63.06 |
| hydroxyleucine | 3-CH | 3.63 70.39 | 3.63 70.49 | 3.63 70.4 | 3.62 70.38 | 3.79 67.98 |
| A5: 2-amino-3- | 4-CH | 1.37 29.53 | 1.39 29.44 | 1.38 29.5 | 1.35 29.35 | 1.10 35.89 |
| hydroxy-4- | | | | | | CH₂ 1.11 |
| methylhexanoic | | | | | | 27.91 |
| acid | | | | | | CH₂ 1.27 |
| | | | | | | 27.91 |
| | 5-Me | 0.77 15.77 | 0.78 15.63 | 0.76 15.6 | 0.74 15.51 | 0.75 12.42 |
| | 6-Me | 0.82 21.65 | 0.83 21.68 | 0.81 21.5 | 0.80 21.37 | 0.77 13.47 |
| | N-Me | 2.85 29.67 | 2.86 29.73 | 2.86 29.7 | 2.81 29.81 | 2.87 29.75 |
| | OH | 5.05 | 5.02 | | | 4.96 |
| F: A1, A3, A5: | >CO | 173.39 | 173.2 | 173.3 | 173.42 | 173.23 |
| 2-amino-4- | 2-CH | 4.71 49.00 | 4.84 49.08 | 4.69 49.0 | 4.83 48.76 | 4.69 49.00 |
| methyl-5- | 3-CH₂ | 1.51 36.48 | 1.38 38.0 | 1.50 36.2 | 1.47 36.12 | 1.49 36.35 |
| hexenoic acid | | 1.59 36.48 | 1.56 38.0 | 1.60 36.2 | | 1.61 36.36 |
| A2: leucine. | 4-CH | 2.01 36.20 | 1.54 25.7 | 2.02 36.0 | 1.78 33.52 | 2.02 36.04 |
| A4: 2-amino-4- | 4 | 5.66 143.11 | 5.65 143.0 | | 0.96 27.47 | 5.66 143.09 |
| methylhexanoic | | | | | 1.14 27.47 | |
| acid | 5 | 4.87 115.73 | 0.84 23.8 | 4.86 115.8 | 0.75 11.77 | 4.86 115.76 |
| | | 5.00 115.73 | | 5.00 115.8 | | 5.00 115.76 |
| | 5-Me | 0.95 21.42 | 0.86 21.5 | 0.93 21.4 | 0.76 19.82 | 0.93 21.45 |
| | NH | 7.83 | 7.82 | 7.82 | 7.78 | 7.76 |
| G: N-methyl- | >CO | 174.32 | 174.25 | 174 | 174.57 | 174.35 |
| alanine | 2-CH | 4.99 51.21 | 5.00 51.22 | 4.99 50.7 | 4.93 51.35 | 4.99 51.25 |
| | 3-Me | 1.26 14.31 | 1.27 14.38 | 1.26 14.3 | 1.25 14.10 | 1.26 14.33 |
| | N-Me | 2.96 31.72 | 3.04 31.79 | 2.96 31.7 | 3.00 31.58 | 2.96 31.75 |

### Stereochemistry of Compounds A1-A5

The absolute configurations of some of the amino acid units were determined by acid hydrolysis followed by derivatisation with Marfey's reagent (Nα-(2,4-dinitro-5-fluorophenyl)-L-alaninamide) and subsequent HPLC analysis). By comparing the derived chromatograms against the HPLC chromatograms derived from enantiomers of the commercially available amino acids N-Me-alanine, isoleucine, pipecolic acid assignments of configuration could be made. Both of the N-Me-alanine units in A3 were found to be of (9-configuration and the isoleucine and pipecolic acid units were (R)-configuration. The derived partial configuration of A3 is depicted in FIG. 3.

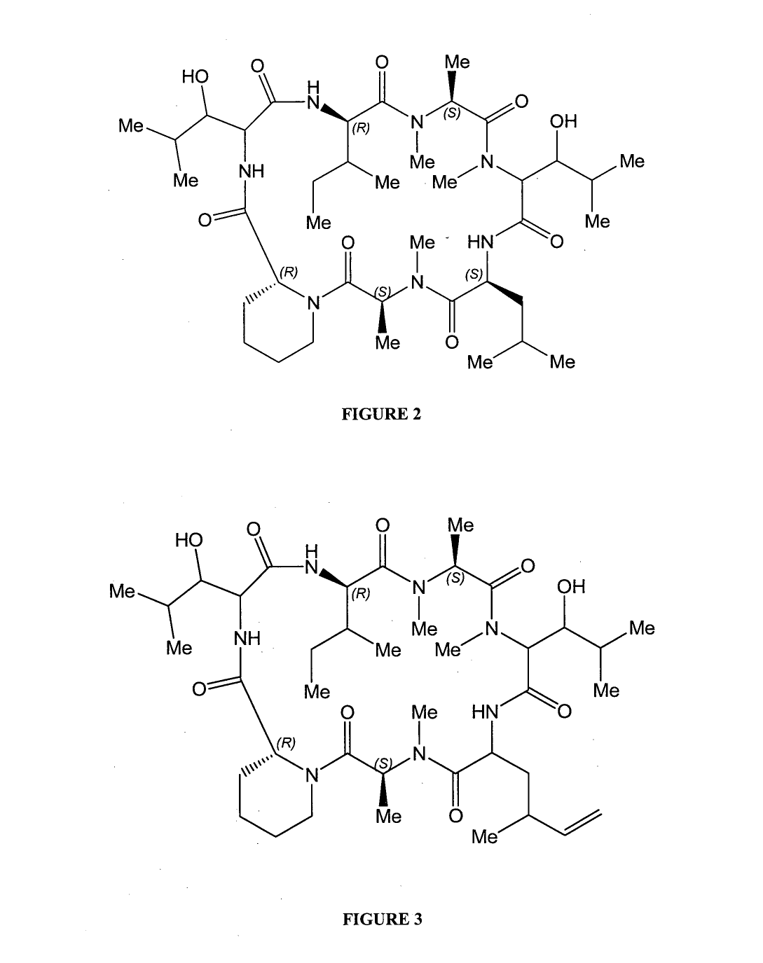

In a similar fashion A2 was hydrolysed and analysed. The only difference between A3 and A2 was a change of 2-amino-4-methyl-5-hexenoic acid to a leucine (amino acid F). The HPLC analysis of the Marfey derivatives of the amino acids from the hydrolysis of A2 and comparison against the reference amino acids indicated A2 also contained pipecolic acid and isoleucine with (R)-configurations, while the two N-Me-alanines and the leucine had (s)-configurations. The partial absolute stereochemistry of A2 is shown in FIG. 2.

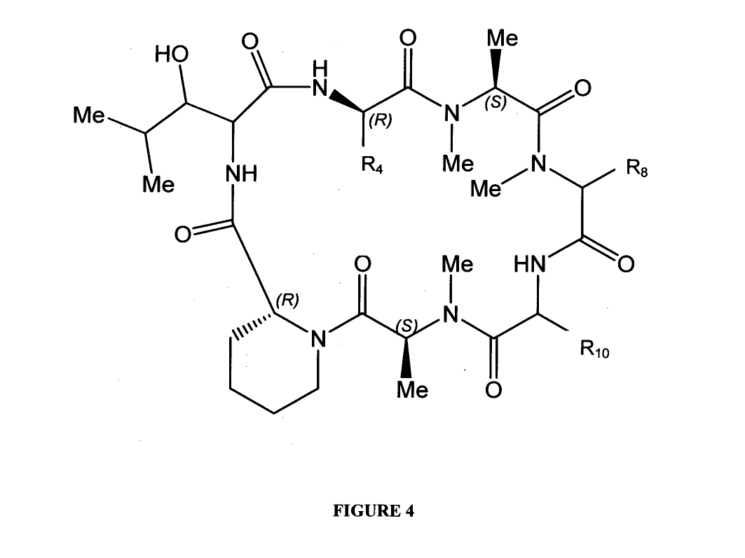

Lack of material prevented the partial assignments of the other three peptides being determined. To obtain the absolute stereochemistry of the peptide series it will be necessary to undertake synthetic studies to obtain the necessary stereoisomers of the remaining amino acids. Based on the findings to date the relative stereochemistry for pipecolic acid, isoleucine and N-Me-alanine will likely be maintained through the series. Therefore, it can be concluded that a compound of Formula Ia may possess the partial stereochemical structure as seen in FIG. 4.

### Biological Activity of Compounds A1-A5

The Compounds A1-A5 were assayed against three cell lines: murine leukemia, P388; human breast cancer, MCF7 (ATCC HTB-22); and human colon cancer, HCT116 (ATCC CCL-247). The results are shown in Table 11.

### Cell Culture

Human breast cancer, MCF7 (ATCC HTB-22) and human colon cancer, HCT116 (ATCC CCL-247) cell lines were all maintained in RPMI 1640 (Sigma), supplemented with 10% Foetal Bovine Serum (FBS, PAA Laboratories). Cells of 80-85% confluence were harvested and plated onto 96-flat bottom well plates for experimental use. In all experiments, cells were incubated in a CO₂ incubator at 37° C. with 5% CO₂ overnight prior to treatment.

### Cytotoxic Assay

Compounds were tested against MCF7 and HCT116 and incubated for 96 h before cytotoxic assay using the MTT (3-(4,5-dimethylthiazole-2-yl)-2,5-diphenyltetrazolium bromide) assay according to Mosmann (J. Immunol Meth. 65 (1983) 55-63). Plates were read using an Elisa plate reader at 520 nm. Data generated was used to plot a dose response curve. Cytotoxic activity was expressed as the mean concentration of extract required to kill 50% of the cell population (IC₅₀).

TABLE 11
Activity of Compound A1-A5 series peptides against the P388 murine
leukemia, HCT116 (ATCC CCL-247) human colon cancer, and
MCF7 (ATCC HFB-22) human breast cancer cell lines

| | IC₅₀ (ng/mL) | | | IC₅₀ (nM) | | |
|---|---|---|---|---|---|---|
| | P388 | HCT116 | MCF7 | P388 | HCT116 | MCF7 |
| Compound A1 | 3 | 4.7 | 6.5 | 3.8 | 5.9 | 8.1 |
| Compound A2 | 45 | 9.3 | 6.6 | 56 | 11.6 | 8.3 |
| Compound A3 | 1 | 0.2 | 0.73 | 1.3 | 0.3 | 0.9 |
| Compound A4 | 19 | 3.1 | 5.7 | 24 | 3.9 | 7.1 |
| Compound A5 | 0.1 | 0.2 | 0.8 | 0.13 | 0.3 | 1.0 |

The data in Table 11 represent a compilation of structure activity relationships with a factor of ˜450 between the least active, Compound A2, and the most active, Compound A5, for the P388 data. There is a high degree of homology across the series with four of the seven amino acids in each peptide being invariant. These are the residues at A (pipecolic acid), B (3-hydroxyleucine), D (N-methylalanine) and G (N-methylalanine). Starting from the most abundant peptide, Compound A3, substitution of amino acid C (isoleucine) with a valine causes a 3-fold decrease in activity. Likewise, a 57-fold decrease in activity occurs if 2-amino-4-methyl-5-hexenoic acid (amino acid F) is replaced by a leucine. Reduction of the vinyl group of 2-amino-4-methyl-5-hexenoic acid (amino acid F) also leads to a reduction in activity (2.4-fold), but if N-methyl-3-hydroxyleucine is replaced with N-methyl-2-amino-3-hydroxy-4-methylhexanoic acid there is a 10-fold increase in activity.

The five heptapeptides in this series are constituted by combinations of five regular amino acids—pipecolic acid, valine, isoleucine and N-methylalanine—and five irregular amino acids—3-hydroxyleucine, N-methyl-3-hydroxyleucine, 2-amino-4-methyl-5-hexenoic acid, 2-amino-4-methylhexanoic acid and 2-amino-3-hydroxy-4-methyl-hexanoic acid in various combinations.

### INDUSTRIAL APPLICATION

It will be appreciated from the discussion above that this invention provides novel bioactive compounds having cytotoxic properties. These compounds may be formulated into pharmaceutical compositions for use in any therapeutic application for which their cytotoxic properties make them appropriate. Such therapeutic applications include anti-cancer treatment.

It is not the intention to limit the scope of the invention to the abovementioned examples only. As would be appreciated by a skilled person in the art, many variations are possible without departing from the scope of the invention.

1. A compound according to Formula I: 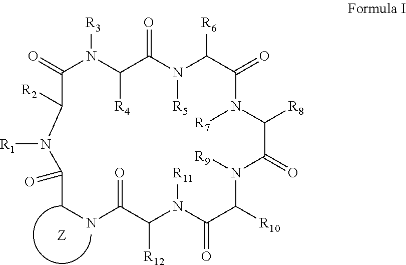
or a pharmaceutically acceptable salt, solvate, hydrate or prodrug derivative thereof, as pure stereoisomers, mixture of isomers, in enol form or tautomeric form, comprising of:
R₁, R₃, R₅, R₇, R₉ and R₁₁, which are each independently selected from the group consisting of —H, alkyl, substituted alkyl and —(C═O)R, where R is selected from the group consisting of alkyl, substituted alkyl, cycloalkyl, substituted cycloalkyl, alkenyl, substituted alkenyl, cycloalkenyl, substituted cycloalkenyl, alkynyl, substituted alkynyl, aryl, substituted aryl, heteroaryl, substituted heteroaryl, heterocyclyl and substituted heterocyclyl;
R₂, R₄, R₈, and R₁₀, which are each independently selected from the group consisting of branched alkyl, substituted branched alkyl, branched alkenyl and substituted branched alkenyl, wherein each branched alkyl, substituted branched alkyl, branched alkenyl and substituted branched alkenyl is aliphatic; and wherein each substituted branched alkyl, substituted branched alkenyl and/or substituted branched alkynyl has 1-3 substituents each independently selected from the group consisting of: —OH, —OR′, —SH, —SR′, —SOR′, —SO₂R′, —NO₂, —NH₂, —NHR′, —N(R′)₂, —NHCOR′, —N(COR′)₂, —NHSO₂R′, —CN, halogen, —C(═O)H, —C(═O)R′, —CO₂H, —CO₂R′, alkyl, alkyl substituted with 1-3 R″, alkenyl, alkenyl substituted with 1-3 R″, alkynyl and alkynyl substituted with 1-3 R″;
R₆ and R₁₂, which are each independently selected from the group consisting of unbranched alkyl, substituted unbranched alkyl, unbranched alkenyl and substituted unbranched alkenyl; wherein each substituted unbranched alkyl, substituted cycloalkyl, substituted unbranched alkenyl, substituted cycloalkenyl, substituted unbranched alkynyl, substituted aryl, substituted heteroaryl, and/or substituted heterocyclyl has 1-3 substituents each independently selected from the group consisting of: —OH, —OR′, —SH, —SR′, —SOR′, —SO₂R′, —NO₂₅—NH₂, —NHR′, —N(R′)₂, —NHCOR′, —N(COR′)₂, —NHSO₂R′, —CN, halogen, —C(═O)H, —C(═O)R′, —CO₂H, —CO₂R′, cycloalkenyl, cycloalkenyl substituted with 1-3 R″, aryl, aryl substituted with 1-3 R″, heterocyclyl, heterocyclyl substituted with 1-3 R″, heteroaryl and heteroaryl substituted with 1-3 R″;
Z ring, which is selected from the group consisting of: 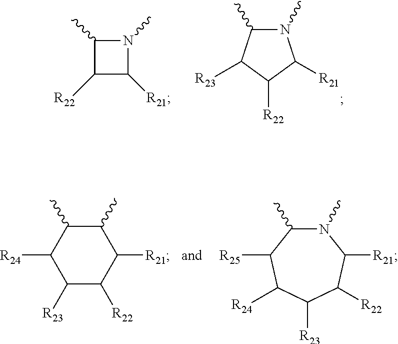
wherein R₂₁, R₂₂, R₂₃, R₂₄ and R₂₅ are each independently selected from the group consisting of: —H, —OH, —OR′, —SH, —SR′, —SOR′, —SO₂R′, ″NO₂, —NH₂, —NHR′, —N(R′)₂, —NHCOR′, —N(COR′)₂, —NHSO₂R′, —CN, halogen, —C(═O)H, —C(═O)R, —CO₂H, —CO₂R′, alkyl, alkyl substituted with 1-3 R″, alkenyl, alkenyl substituted with 1-3 R″, cycloalkenyl, cycloalkenyl substituted with 1-3 R″, alkynyl, alkynyl substituted with 1-3 R″, aryl, aryl substituted with 1-3 R″, heterocyclyl, heterocyclyl substituted with 1-3 R″, heteroaryl and heteroaryl substituted with 1-3 R″; and
wherein each R′ is independently selected from the group consisting of alkyl, alkyl substituted with 1-3 R″, cycloalkyl, cycloalkyl substituted with 1-3 R″, alkenyl, alkenyl substituted with 1-3 R″, cycloalkenyl, cycloalkenyl substituted with 1-3 R″, alkynyl, alkynyl substituted with 1-3 R″, aryl, aryl substituted with 1-3 R″, alkylaryl, alkylaryl substituted with 1-3 R″, heterocyclyl, heterocyclyl substituted with 1-3 R″, heteroaryl and heteroaryl substituted with 1-3 R″; wherein each R″ is independently selected from the group consisting of —OH, —SH, —NO₂, —NH₂, —CN, halogen, —C(═O)H, and —CO₂H.

2. The compound according to claim 1, wherein said alkyl comprises 1 to 6 carbon atoms, or said aryl comprises 3 to 10 carbon atoms, or said cycloalkyl comprises 3 to 6 carbon atoms.

3. The compound according to claim 1, wherein said alkyl is methyl, ethyl, n-propyl, iso-propyl, n-butyl, iso-butyl, sec-butyl, tert-butyl, n-pentyl, 1-methylbutyl, 2-methylbutyl, 3-methylbutyl or 2,2′-dimethylpropyl.

4-5. (canceled)

6. The compound according to claim 1, wherein R₁, R₃ and R₉ are —H.

7. The compound according to claim 1, wherein R₅, R₇, and R₁₁ are alkyl, preferably methyl.

8. The compound according to claim 1, wherein R₄ is branched alkyl, R₂, and R₁₂ are unbranched alkyl and R₂ and R₈ are substituted branched alkyl.

9. The compound according to claim 1, wherein R₆ and R₁₂ are unbranched alkyl, preferably methyl.

10. The compound according to claim 1, wherein R₂ is substituted branched alkyl, preferably 1-hydroxy-2-methylpropyl.

11. The compound according to claim 1, wherein R₄ is branched alkyl, preferably iso-propyl or sec-butyl.

12. The compound according to claim 1, wherein R₈ is substituted branched alkyl, preferably 1-hydroxy-2-methylpropyl or 1-hydroxy-2-methylbutyl.

13. The compound according to claim 1, wherein R₁₀ is branched alkyl, preferably iso-butyl or 2-methylbutyl or R₁₀ is branched alkenyl, preferably 2-methyl-3-butenyl.

14. (canceled)

15. The compound according to claim 1, wherein R₁, R₃ and R₉ are —H; R₅, R₇, and R₁₁ are methyl; R₂ is 1-hydroxy-2-methylpropyl; R₄ is iso-propyl or sec-butyl; R₆ and R₁₂ are methyl; R₈ is 1-hydroxy-2-methylpropyl or 1-hydroxy-2-methylbutyl; R₁₀ is iso-butyl, 2-methylbutyl or 2-methyl-3-butenyl; and the Z ring is:

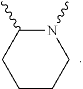

16. The compound according to claim 1, wherein the compound has the Formula Ia: 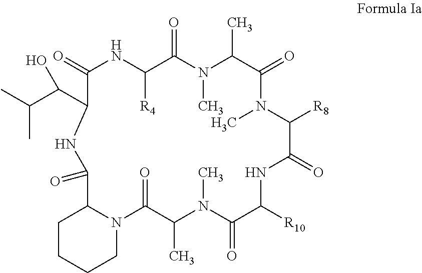
wherein R₄, R₈ and R₁₀ are as defined for Formula I according to claim 1.

17. The compound according to claim 16, wherein R₄ is alkyl, preferably iso-propyl or sec-butyl.

18. The compound according to claim 16, wherein R₈ is substituted alkyl, preferably 1-hydroxy-2-methylpropyl or 1-hydroxy-2-methylbutyl.

19. The compound according to claim 16, wherein R₁₀ is alkyl, preferably iso-butyl or 2-methylbutyl or R₁₀ is alkenyl, preferably 2-methyl-3-butenyl.

20. (canceled)

21. The compound according to claim 16, wherein the compound has a partial stereochemical structure of:

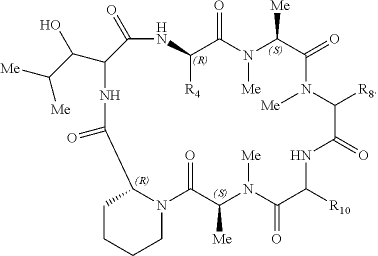

22. The compound according to claim 21, wherein R₄ is sec-butyl, R₈ is 1-hydroxy-2-methylpropyl and R₁₀ is iso-butyl or 2-methyl-3-butenyl.

23-24. (canceled)

25. A method for treatment or prophylaxis of a cancer in a mammal comprising the step of administering a therapeutically effective amount of a compound of claim 1 to the mammal.

26-27. (canceled)

28. A pharmaceutical composition comprising a compound of claim 1 and a pharmaceutically acceptable carrier, diluent or excipient.
